SEQUENCE LISTING (91 sequences)

SEQ ID NO: 1 - protein, 20 aa, Artificial Sequence
  Description of Artificial Sequences; note = synthetic construct
PSSRASSRASSRPRPDDLEI

SEQ ID NO: 2 - protein, 9 aa, Artificial Sequence
  Description of Artificial Sequences; note = synthetic construct
RPRPDDLEI

SEQ ID NO: 3 - protein, 9 aa, Artificial Sequence
  Description of Artificial Sequences; note = synthetic construct
RPRPDDLEV

SEQ ID NO: 4 - protein, 9 aa, Artificial Sequence
  Description of Artificial Sequences; note = synthetic construct
RPRPDDVPV

SEQ ID NO: 5 - protein, 9 aa, Artificial Sequence
  Description of Artificial Sequences; note = synthetic construct
KARSDDLSV

SEQ ID NO: 6 - DNA, 27 bp, Artificial Sequence
  Description of Artificial Sequences; note = synthetic construct
agacctcggcctgatgacctggagatt

SEQ ID NO: 7 - protein, 16 aa, Artificial Sequence
  Description of Artificial Sequences; note = synthetic construct
RQPKIWFPNRRKPWKK

SEQ ID NO: 8 - protein, 36 aa, Artificial Sequence
  Description of Artificial Sequences; note = synthetic construct
RQPKIWFPNRRKPWKKPSSRASSRASSRPRPDDLEI

SEQ ID NO: 9 - protein, 25 aa, Artificial Sequence
  Description of Artificial Sequences; note = synthetic construct
RQPKIWFPNRRKPWKKRPRPDDLEI

SEQ ID NO: 10 - protein, 25 aa, Artificial Sequence
  Description of Artificial Sequences; note = synthetic construct
RQPKIWFPNRRKPWKKRPRPDDLEV

SEQ ID NO: 11 - protein, 25 aa, Artificial Sequence
  Description of Artificial Sequences; note = synthetic construct
RQPKIWFPNRRKPWKKRPRPDDVPV

SEQ ID NO: 12 - protein, 25 aa, Artificial Sequence
  Description of Artificial Sequences; note = synthetic construct
RQPKIWFPNRRKPWKKKARSDDLSV

SEQ ID NO: 13 - DNA, 74 bp, Artificial Sequence
  Description of Artificial Sequences; note = synthetic construct
cggcagcccaagatctggttccccaaccggcggaagccctggaagaagcggcccggcccgacgacctggagatc

SEQ ID NO: 14 - protein, 11 aa, Artificial Sequence
  Description of Artificial Sequences; note = synthetic construct
GRKKRRQRPPQ

SEQ ID NO: 15 - protein, 16 aa, Artificial Sequence
  Description of Artificial Sequences; note = synthetic construct
RQIKIWFQNRRMKWKK

SEQ ID NO: 16 - protein, 16 aa, Artificial Sequence
  Description of Artificial Sequences; note = synthetic construct
RQIAIWFQNRRMKWAA

SEQ ID NO: 17 - protein, 9 aa, Artificial Sequence
  Description of Artificial Sequences; note = synthetic construct
RKKRRQRRR

SEQ ID NO: 18 - protein, 21 aa, Artificial Sequence
  Description of Artificial Sequences; note = synthetic construct
TRSSRAGLQFPVGRVHRLLRK

SEQ ID NO: 19 - protein, 26 aa, Artificial Sequence
  Description of Artificial Sequences; note = synthetic construct
GWTLNSAGYLLGKINKALAALAKKIL

SEQ ID NO: 20 - protein, 18 aa, Artificial Sequence
  Description of Artificial Sequences; note = synthetic construct
KLALKLALKALKAALKLA

SEQ ID NO: 21 - protein, 16 aa, Artificial Sequence
  Description of Artificial Sequences; note = synthetic construct
AAVALLPAVLLALLAP

SEQ ID NO: 22 - protein, 10 aa, Artificial Sequence
  Description of Artificial Sequences; note = synthetic construct
VPMLKPMLKE

SEQ ID NO: 23 - protein, 28 aa, Artificial Sequence
  Description of Artificial Sequences; note = synthetic construct
MANLGYWLLALFVTMWTDVGLCKKRPKP

SEQ ID NO: 24 - protein, 18 aa, Artificial Sequence
  Description of Artificial Sequences; note = synthetic construct
LLIILRRRIRKQAHAHSK

SEQ ID NO: 25 - protein, 21 aa, Artificial Sequence
  Description of Artificial Sequences; note = synthetic construct
KETWWETWWTEWSQPKKKRKV

SEQ ID NO: 26 - protein, 18 aa, Artificial Sequence
  Description of Artificial Sequences; note = synthetic construct
RGGRLSYSRRRFSTSTGR

SEQ ID NO: 27 - protein, 15 aa, Artificial Sequence
  Description of Artificial Sequences; note = synthetic construct
SDLWEMMMVSLACQY

SEQ ID NO: 28 - protein, 12 aa, Artificial Sequence
  Description of Artificial Sequences; note = synthetic construct
TSPLNIHNGQKL

SEQ ID NO: 29 - protein, 19 aa, Artificial Sequence
  Description of Artificial Sequences; note = synthetic construct
PSRASSRASSRPRPDDLEI

SEQ ID NO: 30 - protein, 20 aa, Artificial Sequence
  Description of Artificial Sequences; note = synthetic construct
GSNKSTASSKSPDPKNSVWI

SEQ ID NO: 31 - protein, 20 aa, Artificial Sequence
  Description of Artificial Sequences; note = synthetic construct
GSNKSSASSKSGDGKNSVWI

SEQ ID NO: 32 - protein, 21 aa, Artificial Sequence
  Description of Artificial Sequences; note = synthetic construct
GRASKASRASSGRARPEDLAI

SEQ ID NO: 33 - protein, 13 aa, Artificial Sequence
  Description of Artificial Sequences; note = synthetic construct
GSASSRDGKTVWI

SEQ ID NO: 34 - protein, 19 aa, Artificial Sequence
  Description of Artificial Sequences; note = synthetic construct
PRVSVPNFGRTQSSDSAYV

SEQ ID NO: 35 - protein, 19 aa, Artificial Sequence
  Description of Artificial Sequences; note = synthetic construct
PRMSMPNFGRTQSSDSAYV

SEQ ID NO: 36 - protein, 21 aa, Artificial Sequence
  Description of Artificial Sequences; note = synthetic construct
PRAGSEKGSASSRDGKTTVWI

SEQ ID NO: 37 - protein, 23 aa, Artificial Sequence
  Description of Artificial Sequences; note = synthetic construct
GYHSDKRRLSKASSKARSDDLSV

SEQ ID NO: 38 - protein, 19 aa, Artificial Sequence
  Description of Artificial Sequences; note = synthetic construct
PLSRLSKASSRARSDDLTV

SEQ ID NO: 39 - protein, 22 aa, Artificial Sequence
  Description of Artificial Sequences; note = synthetic construct
PNHVVSLTNNLIGRRVPTDLQI

SEQ ID NO: 40 - protein, 29 aa, Artificial Sequence
  Description of Artificial Sequences; note = synthetic construct
PSCVSSSAVLTTICSSDQVVPVGLSSFYM

SEQ ID NO: 41 - protein, 21 aa, Artificial Sequence
  Description of Artificial Sequences; note = synthetic construct
GRSSKASKSSGGRARAADLAI

SEQ ID NO: 42 - protein, 17 aa, Artificial Sequence
  Description of Artificial Sequences; note = synthetic construct
LCYLLIRYCSGKSKKPV

SEQ ID NO: 43 - protein, 18 aa, Artificial Sequence
  Description of Artificial Sequences; note = synthetic construct
GQKPPSRPSSSASKKQYV

SEQ ID NO: 44 - protein, 19 aa, Artificial Sequence
  Description of Artificial Sequences; note = synthetic construct
SSRASSRASSRPRPDDLEV

SEQ ID NO: 45 - protein, 9 aa, Artificial Sequence
  Description of Artificial Sequences; note = synthetic construct
RPKPDDLEI

SEQ ID NO: 46 - protein, 19 aa, Artificial Sequence
  Description of Artificial Sequences; note = synthetic construct
SSRASSRASSRPKPDDLEI

SEQ ID NO: 47 - protein, 9 aa, Artificial Sequence
  Description of Artificial Sequences; note = synthetic construct
RPKPDDLDI

SEQ ID NO: 48 - protein, 19 aa, Artificial Sequence
  Description of Artificial Sequences; note = synthetic construct
SSRASSRASSRPRPDDLDI

SEQ ID NO: 49 - protein, 19 aa, Artificial Sequence
  Description of Artificial Sequences; note = synthetic construct
SSRASTRASSRPRPDDLEI

SEQ ID NO: 50 - protein, 9 aa, Artificial Sequence
  Description of Artificial Sequences; note = synthetic construct
RPRPEDLEI

SEQ ID NO: 51 - protein, 19 aa, Artificial Sequence
  Description of Artificial Sequences; note = synthetic construct
SSRASSRASSRPRPEDLEI

SEQ ID NO: 52 - protein, 9 aa, Artificial Sequence
  Description of Artificial Sequences; note = synthetic construct
GDGKNSVWV

SEQ ID NO: 53 - protein, 23 aa, Artificial Sequence
  Description of Artificial Sequences; note = synthetic construct
SKAGSNKSTASSKSGDGKNSVWV

SEQ ID NO: 54 - protein, 18 aa, Artificial Sequence
  Description of Artificial Sequences; note = synthetic construct
GQKPPSRPSSSASKKLYV

SEQ ID NO: 55 - protein, 24 aa, Artificial Sequence
  Description of Artificial Sequences; note = synthetic construct
RQPKIWFPNRRKPWKIELDDPRPR

SEQ ID NO: 56 - protein, 20 aa, Artificial Sequence
  Description of Artificial Sequences; note = synthetic construct
GRKKRRQRPPQRPRPDDLEI

SEQ ID NO: 57 - protein, 25 aa, Artificial Sequence
  Description of Artificial Sequences; note = synthetic construct
RQIKIWFQNRRMKWKKRPRPDDLEI

SEQ ID NO: 58 - protein, 25 aa, Artificial Sequence
  Description of Artificial Sequences; note = synthetic construct
RQIAIWFQNRRMKWAARPRPDDLEI

SEQ ID NO: 59 - protein, 18 aa, Artificial Sequence
  Description of Artificial Sequences; note = synthetic construct
RKKRRQRRRRPRPDDLEI

SEQ ID NO: 60 - protein, 30 aa, Artificial Sequence
  Description of Artificial Sequences; note = synthetic construct
TRSSRAGLQFPVGRVHRLLRKRPRPDDLEI

SEQ ID NO: 61 - protein, 35 aa, Artificial Sequence
  Description of Artificial Sequences; note = synthetic construct
GWTLNSAGYLLGKINKALAALAKKILRPRPDDLEI

SEQ ID NO: 62 - protein, 27 aa, Artificial Sequence
  Description of Artificial Sequences; note = synthetic construct
KLALKLALKALKAALKLARPRPDDLEI

SEQ ID NO: 63 - protein, 25 aa, Artificial Sequence
  Description of Artificial Sequences; note = synthetic construct
AAVALLPAVLLALLAPRPRPDDLEI

SEQ ID NO: 64 - protein, 19 aa, Artificial Sequence
  Description of Artificial Sequences; note = synthetic construct
VPMLKPMLKERPRPDDLEI

SEQ ID NO: 65 - protein, 37 aa, Artificial Sequence
  Description of Artificial Sequences; note = synthetic construct
MANLGYWLLALFVTMWTDVGLCKKRPKPRPRPDDLEI

SEQ ID NO: 66 - protein, 27 aa, Artificial Sequence
  Description of Artificial Sequences; note = synthetic construct
LLIILRRRIRKQAHAHSKRPRPDDLEI

SEQ ID NO: 67 - protein, 30 aa, Artificial Sequence
  Description of Artificial Sequences; note = synthetic construct
KETWWETWWTEWSQPKKKRKVRPRPDDLEI

SEQ ID NO: 68 - protein, 27 aa, Artificial Sequence
  Description of Artificial Sequences; note = synthetic construct
RGGRLSYSRRRFSTSTGRRPRPDDLEI

SEQ ID NO: 69 - protein, 24 aa, Artificial Sequence
  Description of Artificial Sequences; note = synthetic construct
SDLWEMMMVSLACQYRPRPDDLEI

SEQ ID NO: 70 - protein, 21 aa, Artificial Sequence
  Description of Artificial Sequences; note = synthetic construct
TSPLNIHNGQKLRPRPDDLEI

SEQ ID NO: 71 - protein, 120 aa, Artificial Sequence
  Description of Artificial Sequences; note = synthetic construct
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 72 - protein, 120 aa, Artificial Sequence
  Description of Artificial Sequences; note = synthetic construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 73 - protein, 120 aa, Artificial Sequence
  Description of Artificial Sequences; note = synthetic construct
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 74 - protein, 120 aa, Artificial Sequence
  Description of Artificial Sequences; note = synthetic construct
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 75 - protein, 120 aa, Artificial Sequence
  Description of Artificial Sequences; note = synthetic construct
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 76 - protein, 120 aa, Artificial Sequence
  Description of Artificial Sequences; note = synthetic construct
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 77 - protein, 239 aa, Artificial Sequence
  Description of Artificial Sequences; note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - DNA, 60 bp, Artificial Sequence
  Description of Artificial Sequences; note = synthetic construct
ccctcctcccgggcctcctcccgggcctcctcccggccccggcccgacgacctggagatc

SEQ ID NO: 79 - DNA, 27 bp, Artificial Sequence
  Description of Artificial Sequences; note = synthetic construct
cggccccggcccgacgacctggagatc

SEQ ID NO: 80 - DNA, 27 bp, Artificial Sequence
  Description of Artificial Sequences; note = synthetic construct
cggccccggcccgacgacctggaggtg

SEQ ID NO: 81 - DNA, 27 bp, Artificial Sequence
  Description of Artificial Sequences; note = synthetic construct
cggccccggcccgacgacgtgcccgtg

SEQ ID NO: 82 - DNA, 27 bp, Artificial Sequence
  Description of Artificial Sequences; note = synthetic construct
aaggcccggtccgacgacctgtccgtg

SEQ ID NO: 83 - DNA, 48 bp, Artificial Sequence
  Description of Artificial Sequences; note = synthetic construct
cggcagcccaagatctggttccccaaccggcggaagccctggaagaag

SEQ ID NO: 84 - DNA, 108 bp, Artificial Sequence
  Description of Artificial Sequences; note = synthetic construct
cggcagcccaagatctggttccccaaccggcggaagccctggaagaagccctcctcccgggcctcctcccgggcctcctcccggccccggcccgacgacctggagatc

SEQ ID NO: 85 - DNA, 75 bp, Artificial Sequence
  Description of Artificial Sequences; note = synthetic construct
cggcagcccaagatctggttccccaaccggcggaagccctggaagaagcggccccggcccgacgacctggagatc

SEQ ID NO: 86 - DNA, 75 bp, Artificial Sequence
  Description of Artificial Sequences; note = synthetic construct
cggcagcccaagatctggttccccaaccggcggaagccctggaagaagcggccccggcccgacgacctggaggtg

SEQ ID NO: 87 - DNA, 75 bp, Artificial Sequence
  Description of Artificial Sequences; note = synthetic construct
cggcagcccaagatctggttccccaaccggcggaagccctggaagaagcggccccggcccgacgacgtgcccgtg

SEQ ID NO: 88 - DNA, 75 bp, Artificial Sequence
  Description of Artificial Sequences; note = synthetic construct
cggcagcccaagatctggttccccaaccggcggaagccctggaagaagaaggcccggtccgacgacctgtccgtg

SEQ ID NO: 89 - protein, 20 aa, Artificial Sequence
  Description of Artificial Sequences; note = synthetic construct
PCSRASSRMSSRARPDDLDV

SEQ ID NO: 90 - protein, 19 aa, Artificial Sequence
  Description of Artificial Sequences; note = synthetic construct
PRVSVPNFGRTQSSDSAYV

SEQ ID NO: 91 - protein, 26 aa, Artificial Sequence
  Description of Artificial Sequences; note = synthetic construct
  MOD_RES (1)..(1): Xaa is biotinylated
  MOD_RES (1)..(1): Xaa is L-2-aminohexanoic acid
XRQPKIWFPNRRKPWKKRPRPDDLEI

(19) United States
(12) Patent Application Publication
(10) Pub. No.: US 2021/0361741 A1
(43) Pub. Date: Nov. 25, 2021

(54) NANOPARTICLE FORMULATIONS AND METHODS OF USE FOR ALPHA CONNEXIN C-TERMINAL PEPTIDES

(71) Applicant: FIRSTSTRING RESEARCH, INC., Mt. Pleasant, SC (US)

(72) Inventors: Christina L. GREK, Mt. Pleasant, SC (US); Gautam S. GHATNEKAR, Mt. Pleasant, SC (US)

(21) Appl. No.: 17/275,577

(22) PCT Filed: Sep. 12, 2019
(86) PCT No.: PCT/US19/50822

§ 371 Date: Mar. 11, 2021

Related U.S. Application Data
(60) Provisional application No. 62/730,116, filed on Sep. 12, 2018.

(51) Int. Cl. A61K 38/17; A61K 9/16; A61K 9/00; A61K 47/02; A61K 31/495; A61K 31/196; A61K 31/675; A61K 31/17; A61K 38/14; A61K 38/50; A61K 31/475; A61K 31/337; A61K 31/282; A61K 31/704; A61P 35/00

(52) U.S. Cl. CPC A61K 38/177; A61P 35/00; A61K 9/1635; A61K 9/0014; A61K 47/02; A61K 9/1694; A61K 31/495; A61K 31/196; A61K 31/675; A61K 31/17; A61K 38/14; A61K 38/50; A61K 31/475; A61K 31/337; A61K 31/282; A61K 31/704; A61K 9/1647

(57) ABSTRACT

The present disclosure relates to nanoparticle formulations and methods of use for alpha connexin c-terminal peptides. Methods of making and methods of use, for example in the treatment of cancer, are also provided.

### CROSS-REFERENCE TO RELATED APPLICATIONS

This application claims the benefit of priority to U.S. Provisional Application No. 62/730,116, filed on Sep. 12, 2018, which is hereby incorporated by reference in its entirety.

### STATEMENT REGARDING FEDERALLY FUNDED RESEARCH

This invention was made with government support awarded by Grant No. R43 CA195937 awarded by the National Institutes of Health, and Grant No. R43 CA195937, awarded by the National Institutes of Health. The government has certain rights in this invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: FIRS_008_01WO_SeqList_ST25.txt, date recorded: Feb. Sep. 12, 2019, file size 34 kilobytes).

### FIELD OF DISCLOSURE

The present disclosure relates to αCT1 peptide-containing nanoparticle formulations, and to the treatment of cancer and other indications with αCT1-containing nanoparticle formulations.

### BACKGROUND

In vivo susceptibility to physical and chemical alteration (i.e. denaturation, aggregation, oxidation, hydrolysis, etc.) has hindered the therapeutic development of peptides and proteins. Biodegradable nanoparticles offer the opportunity to achieve sustained therapeutic drug delivery in addition to offering drug targeting and reduced off-target side effects.¹⁻³ Due to their biocompatibility and biodegradability, polymeric nanoparticles, such as poly(lactic-co-glycolic acid) (PLGA) nanoparticles, have been used for controlled release of various small molecule drugs.^{4, 5} Sustained drug release is especially useful for chronic diseases, such as non-healing ulcers, and other ailments such as cancer that may take weeks or longer to heal or resolve and may require repetitive therapeutic intervention.⁵

αCT1 (also referred to as αCT1 or ACT1) is a synthetic C-terminus connexin43 mimetic peptide drug currently in clinical trials for the treatment of chronic wounds⁶⁻⁸ and animal trials for the treatment of glioblastoma (brain cancer).⁹ Current treatment paradigms involve multiple applications or administrations.

There is a need in the art for biocompatible, sustained-release peptide nanoparticle formulations and new methods for readily preparing the same. In addition, there is a need in the art for biocompatible, sustained-release peptide nanoparticle formulations that can be intravenously administered.

### SUMMARY OF THE DISCLOSURE

In some embodiments, the present disclosure provides a composition comprising one or more nanoparticles, wherein the nanoparticles comprise one or more biodegradable or biocompatible polymers and a therapeutically effective amount of a peptide comprising an amino acid sequence according to SEQ ID NO: 1. In some embodiments, the peptide further comprises a cellular internalization sequence (e.g. an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol). In some embodiments, the peptide comprises an amino acid sequence according to SEQ ID NO: 2.

In some embodiments, the one or more biodegradable or biocompatible polymers are PLGA. In some embodiments, the one or more biodegradable or biocompatible polymers are PLGA and PVA. In some embodiments, the PLGA has a Mw from about 4,000 to about 240,000 Da, for example from about 7,000 to about 17,000 Da. In some embodiments, the PVA has a Mw from about 8,000 to about 186,000 Da, for example from about 13,000 to about 23,000 Da. In some embodiments, the amount of PVA is between about 0.05% (w/v) % to about 5% (w/v). In some embodiments, the amount of PLGA is between about 2% (w/v) to about 10% (w/v). In some embodiments, the average diameter of the nanoparticles is between about 10 nm and about 1000 nm (e.g. between about 100 nm and about 200 nm). In some embodiments, the average amount of the peptide comprising an amino acid sequence according to SEQ ID NO: 1 or SEQ ID NO: 2 is at least about 500 ng per mg of the nanoparticle composition. In some embodiments, the nanoparticles have a surface charge characterized by a zeta potential of between about 0 mV to about −30 mV. In some embodiments, the nanoparticles have a polydispersity index (PDI) of from about 0.120 to about 0.350.

In some embodiments, the present disclosure provides a pharmaceutical formulation comprising the nanoparticle composition of the present disclosure and one or more pharmaceutically acceptable carriers or excipients. In some embodiments, the formulation may be a liquid formulated for injection. In some embodiments, the formulation is in the form of an aerosol, cream, foam, emulsion, gel, liquid, lotion, patch, powder, solid, spray, or any combinations thereof.

In some embodiments, the present disclosure provides a topical formulation comprising the nanoparticle composition of the present disclosure. In some embodiments, the topical formulation further comprises hydroxyethylcellulose gel.

In some embodiments, the present disclosure provides a method of making the nanoparticle composition of the present disclosure, comprising the steps of

(a) combining a first solution comprising one or more biodegradable or biocompatible polymers dissolved in an organic solvent with a second solution comprising an amino acid sequence according to SEQ ID NO: 1 or SEQ ID NO: 2 dissolved in a first aqueous solvent;

(b) emulsifying the mixture of step (a);

(c) adding the emulsion of step (b) to a second solution comprising one or more biodegradable or biocompatible polymers dissolved in a second aqueous solvent;

(d) removing the organic solvent; and

(e) optionally purifying the product of (d).

In some embodiments, the method further comprises the step of (f) freezing and/or lyophilizing the product of (d) or (e). In some embodiments, the biodegradable or biocompatible polymers of step (a) is PLGA. In some embodiments, the biodegradable or biocompatible polymers of step (c) further comprises PVA.

In some embodiments, the present disclosure provides a method of making the nanoparticle composition of the present disclosure, comprising

(a) providing
i. an amino acid sequence according to SEQ ID NO: 1 or SEQ ID NO: 2 and one or more biodegradable or biocompatible polymers dissolved in an water-miscible organic solvent; and
ii. an anti-solvent;

(b) mixing amino acid sequence according to SEQ ID NO: 1 or SEQ ID NO: 2 and one or more biodegradable or biocompatible polymers dissolved in the water-miscible organic solvent with the anti-solvent, such that the nanoparticle composition is formed; and

(c) optionally purifying the product of (b).

In some embodiments, the method further comprises the step of (d) freezing and/or lyophilizing the product of (b) or (c). In some embodiments, the biodegradable or biocompatible polymers of step (a) is PLGA. In some embodiments, the biodegradable or biocompatible polymers of step (a) further comprises PVA.

In some embodiments, the present disclosure provides a method of manufacturing a topical formulation comprising:

a) mixing propylene glycol, glycerin, methylparaben and propylparaben until the parabens are completely dissolved;

b) separately mixing purified water, EDTA, monobasic sodium phosphate, dibasic sodium phosphate and D-mannitol until a clear solution is obtained;

c) adding the solution from a) to the solution from b), rinsing the container of the solution from a) with purified water, adding the rinse to the combined solutions, and mixing until the combined solutions are visually homogeneous;

d) with homogenization mixing, adding hydroxyethyl cellulose into the combined solutions of c) and mixing until the polymer is fully dispersed;

e) separately mixing purified water with an amino acid sequence according to SEQ ID NO: 1 or SEQ ID NO: 2 until the peptide is completely dissolved;

f) adding the solution from e) to the solution of d), rinsing the container of the solution from e) with purified water, adding the rinse to the combined solutions, and mixing until the combined solution are homogeneous.

In some embodiments, the present disclosure provides a method of treating cancer in a patient in need thereof wherein the method comprises, administering to the patient a therapeutically effective amount of the pharmaceutical formulation of the present disclosure. In some embodiments, the cancer is glioma (e.g. glioblastoma). In some embodiments, the pharmaceutical formulation is administered by intratumoral injection. In some embodiments, the method further comprises administering a chemotherapeutic agent (e.g. TMZ). In some embodiments, the chemotherapeutic agent is not administered concomitantly with the pharmaceutical formulations (e.g. the chemotherapeutic agent is administered on a different day than the pharmaceutical formulation). In some embodiments, the peptide-nanoparticle compositions provided herein provide an unexpected superior clinical effect in cancer treatment relative to the same peptide that is not associated with a nanoparticle. Thus, in some embodiments, the peptide-nanoparticle compositions provided herein provide an unexpected superior clinical benefit in a glioma relative to the naked peptide. In some embodiments, the peptide-nanoparticle compositions provided herein exhibit superior drug release profiles and/or particle characteristics that result in significantly improved outcomes in cancer patients. In some embodiments, the methods produce peptide-nanoparticles having uniform particle size and little to no nanoparticle agglomeration.

In some embodiments, the present disclosure provides a method of treating a chronic wound in a subject, comprising administering to the subject the topical formulation of the present disclosure, wherein the formulation is administered in a dosing regimen effective for the treatment of the chronic wound (e.g. daily or weekly). In some embodiments, the chronic wound is an ulcer (e.g. a lower extremity ulcer). In some embodiments, the chronic wound is selected from the group consisting of venous leg ulcers, diabetic foot ulcers, and pressure ulcers. In some embodiments, the peptide-nanoparticle compositions provided herein provide an unexpected superior clinical effect in treating chronic wounds. In some embodiments, the peptide-nanoparticle compositions provided herein exhibit superior drug release profiles in the treatment of wounds that results in a superior clinical effect relative to previously known wound treatment therapies. In some embodiments, the peptide-nanoparticle compositions provided herein exhibit particle characteristics that result in significantly improved outcomes in wound treatment.

Other objects, features and advantages of the present disclosure will become apparent from the following detailed description. It should be understood, however, that the detailed description and the specific examples, while indicating preferred embodiments, are given by way of illustration only, since various changes and modifications within the spirit and scope of the disclosure will become apparent to those skilled in the art from this detailed description.

### BRIEF DESCRIPTION OF THE DRAWINGS

FIG. 1 shows a graph of dynamic light scattering peaks for particle size optimization using BSA as a model drug.

FIG. 2 shows percent release of rhodamine B from PLGA-NPs over time.

FIG. 3 shows A) Change in particle diameter with and without freezing when cryoprotectants are not in use. B) Particle diameter after freezing in relation to amount of cryoprotectant used. When 1% sucrose (fast or slow freeze) or 1% trehalose (slow freeze) is added, particle sizes achieved are near the size of unfrozen NPs. Trehalose and sucrose added at concentrations of 15% increased particle size to greater than freezing the particles slowly without any cryoprotectant. Error bars are the standard deviation of at least three samples.

FIG. 4 shows A) Graph of cumulative release of αCT1 from nanoparticles over time as a function of percentage of total drug encapsulated, as measured via sandwich ELISA assay. B) Particle diameter (Z-average) measured by DLS during the degradation of αCT1 nanoparticles and the polydispersity index of the nanoparticles, indicating homogeneity of particle size. SEM images of αCT1-NPs at (C and D) Day 1, (E and F) Day 7, and (G and H) Day 21. Scale bars are 1 μm (C, E, and F) and 100 nm (D, F, and H).

FIG. 5 shows A) Cellular uptake of varying concentration of RhB-NPs to determine optimal concentration for remaining cell studies. Cells incubated overnight before imaging. B) Release of RhB from NPs incorporated into VTC-037 GSCs over three weeks. BF: Bright Field, Fluo: Fluorescence. Scale bar: 200 μm.

FIG. 6 shows incorporation of RhB-NPs into GSCs at 37° C. and 4° C. after incubation for 1 hr. BF: Bright Field, Fluo: Fluorescence. Scale bar: 200 μm.

FIG. 7 shows cellular uptake of RhB-NPs. Scale bar: 20 μm.

FIG. 8 shows cellular uptake of RhB- and αCT1-NPs. Scale bar: 20 μm.

FIG. 9 shows a graphical representation of.

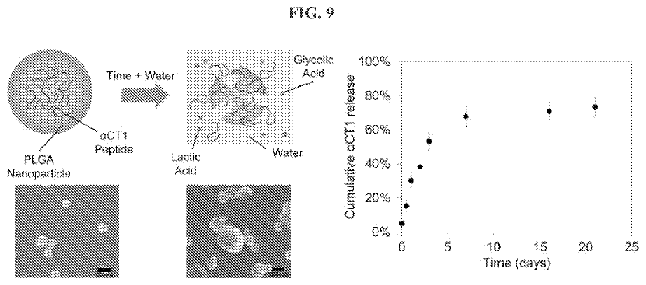

FIG. 10 shows an αCT1-NP flash nanoprecipitation protocol modified from Gindy, M., et al., Langmuir 2008, 24 (1), 83-90., 24 (1), 83-90.

FIG. 11 shows SEM analyses of αCT1-PLGA-NPs synthesized by A.) Double emulsion and B.) Flash nanoprecipitation using a 4-jet mixer. Particle size and morphology are comparable between techniques.

FIG. 12 shows A.) RhodB-NPs were evaluated for release kinetics. No change in NP morphology was detected over time and rhodamine B was detected and 36 days. B.) Double Emulsion or Flash Nanoprecipitation synthesized nanoparticles were redispersed in PBS solution at a concentration of 1 mg/mL and degraded at 37° C. to analyze αCT1 release. Flash nanoprecipiation showed less of an early burst release of αCT1.

FIG. 13 shows that LnN229/GSCs efficiently take up RhodB-NPs (red) and could be detected 24 hours after addition in the cytoplasm.

FIG. 14 shows VTC-037 (primary cells isolated from glioblastoma patient) efficiently take up RhodB-NPs (red) and could be detected in vitro for >21 days. BF=bright field. Fluo=florescence microscopy.

FIG. 15 shows that Human GBM cells efficiently take up αCT1 released by αCT1-NP. αCT1 could be detected in vitro for >4 days. Cells were exposed to αCT1-NP for 24 hours before media was removed. Cell were blocked and permeabilized with PBS/BSA 3%/Triton 0.1% before staining using Streptavidin Alexa Fluor 647 conjugate to detect Biot-αCT1, an antibody against the CT of Cx43 (Sigma #6219) and a secondary goat anti-rabbit antibody conjugated to Alexa Fluor 488, and DAPI to stain the nuclei.

FIG. 16 shows that treatment with αCT1-NP significantly reduced tumor volume when used in combination with TMZ in a GBM xenograft mouse model. Treatment with TMZ alone has no effect, with tumors continuing to grow.

### DETAILED DESCRIPTION

αCT1 is a synthetic C-terminus connexin43 mimetic peptide drug currently in clinical trials for the treatment of chronic wounds⁶⁻⁸ and animal trials for the treatment of glioblastoma (brain cancer).⁹ In the treatment of chronic wounds, the current treatment paradigm involves multiple topical applications over the course of healing. The development of a sustained release alpha connexin peptide formulation could help reduce overall treatment costs and increase patient compliance. In addition, the development of a sustained release alpha connexin peptide formulation that can be administered intratumorally or intravenously would improve the applicability of the alpha connexin peptide properties to various cancer therapies. The peptide has successfully been encapsulated in previous work using alginate microparticles via an electrospray method, resulting in modest drug release profiles of about 24 hours.^{10,11} Given that the therapeutic application of αCT1 may include conveyance within the narrow extracellular spaces of brain tissue (for glioblastoma treatment)⁹ or intravenous delivery, a nanoparticle release system may be a preferred mode of drug delivery compared with microparticles or other bulkier release methods. The present disclosure provides biocompatible, sustained-release αCT1 nanoparticle formulations and methods of making such formulations.

As used above, and throughout this disclosure, the following terms, unless otherwise indicated, shall be understood to have the following meanings. If a term is missing, the conventional term as known to one skilled in the art controls.

As used herein, the terms “including,” “containing,” and “comprising” are used in their open, non-limiting sense.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

A “patient” is a mammal, e.g., a human, mouse, rat, guinea pig, dog, cat, horse, cow, pig, or non-human primate, such as a monkey, chimpanzee, baboon or rhesus. “Patient” includes both human and animals.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The terms “effective amount” or “therapeutically effective amount” when used in connection with a compound refer to a sufficient amount of the compound to provide the desired biological result. That result can be reduction and/or alleviation of the signs, symptoms, or causes of a disease, or any other desired alteration of a biological system. For example, an “effective amount” for therapeutic use is the amount of the composition comprising a compound as disclosed herein required to provide a clinically significant decrease in a disease. An appropriate “effective amount” in any individual case may be determined by one of ordinary skill in the art using routine experimentation. Thus, the expression “effective amount” generally refers to the quantity for which the active substance has therapeutic effects.

As used herein, the terms “treat” or “treatment” are synonymous with the term “prevent” and are meant to indicate a postponement of development of diseases, preventing the development of diseases, and/or reducing severity of such symptoms that will or are expected to develop. Thus, these terms include ameliorating existing disease symptoms, preventing additional symptoms, ameliorating or preventing the underlying causes of symptoms, inhibiting the disorder or disease, e.g., arresting the development of the disorder or disease, relieving the disorder or disease, causing regression of the disorder or disease, relieving a condition caused by the disease or disorder, or stopping or alleviating the symptoms of the disease or disorder.

The term “disorder” is used in this disclosure to mean, and is used interchangeably with, the terms disease, condition, or illness, unless otherwise indicated.

By using the terms “pharmaceutically acceptable” or “pharmacologically acceptable” it is intended to mean a material which is not biologically, or otherwise, undesirable—the material may be administered to an individual without causing any substantially undesirable biological effects or interacting in a deleterious manner with any of the components of the composition in which it is contained.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject. Excipients should be selected on the basis of compatibility and the release profile properties of the desired dosage form. Exemplary carrier materials include, e.g., binders, suspending agents, disintegration agents, filling agents, surfactants, solubilizers, stabilizers, lubricants, wetting agents, diluents, spray-dried dispersions, and the like.

The term “pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover, John E., Remington's Pharmaceutical Sciences, Mack Publishing Co., Easton, Pa. 1975.

As used herein, the term “subject” encompasses mammals and non-mammals. Examples of mammals include, but are not limited to, any member of the class Mammalia: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present disclosure, the mammal is a human.

The terms “administered”, “administration”, or “administering” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body, including an animal, in need of treatment by bringing such individual in contact with, or otherwise exposing such individual to, such compound.

As used herein, the term “delivering” means providing an entity to a destination. For example, delivering a therapeutic and/or prophylactic to a subject may involve administering a nanoparticle of the present disclosure to the subject (e.g., by a topical, intravenous, intramuscular, intradermal, or subcutaneous route). Administration of a nanoparticle to a mammal or mammalian cell may involve contacting one or more cells with the nanoparticle.

Encapsulation means the amount of the drug (e.g. therapeutically effective amount of an alpha connexin polypeptide, such as, for example, a peptide comprising an amino acid sequence according to SEQ ID NO: 1 or SEQ ID NO 2), loaded, associated, bound or otherwise attached to the nanoparticles. In general, the ability of nanoparticles to encapsulate drug is expressed in % of the drug's starting amount. Thus, the optimal encapsulation percentage 100% is achieved where all drug is encapsulated in nanoparticles.

As used herein, “encapsulation efficiency” refers to the amount of a therapeutic that becomes part of a nanoparticle, relative to the initial total amount of therapeutic used in the preparation of the nanoparticle. For example, if 97 mg of therapeutic are encapsulated in a NP out of a total 100 mg of therapeutic initially provided to the composition, the encapsulation efficiency may be given as 97%. As used herein, “encapsulation” may refer to complete, substantial, or partial enclosure, confinement, surrounding, or encasement.

The term % (w/v) refers to the percentage in weight per unit volume, thus 2% PLGA (w/v) refers to the initial amount of PLGA added to the solvent (i.e. 2 grams of PLGA added to 100 mL of solvent) to form the nanoparticles of the present disclosure.

In this study, nanoparticles were prepared using a variety of methods for the encapsulation and controlled release of the synthetic peptide drug αCT1.

### Alpha Connexin Polypeptides

Connexins are the sub-unit protein of the gap junction channel which is responsible for intercellular communication (Goodenough and Paul, 2003). Based on patterns of conservation of nucleotide sequence, the genes encoding Connexin proteins are divided into two families termed the alpha and beta Connexin genes.

Disclosed herein are compositions comprising a polypeptide comprising a carboxy-terminal amino acid sequence of an alpha Connexin. The C-terminal amino acid sequence of alpha Connexin may be referred to, herein, as an alpha Connexin carboxy-Terminal or ACT polypeptide.

In some aspects, the compositions disclosed herein comprise a polypeptide, which comprises 4 to 30 amino acids from the C-terminus of the alpha Connexin. For example, the polypeptide may comprise 4, 5, 6, 7, 8, 9, 10, 11, 12, 13, 14, 15, 16, 17, 18, 19, 20, 21, 22, 23, 24, 25, 26, 27, 28, 29, 30 amino acids from the C-terminus of the alpha Connexin. In some aspects, the polypeptide does not comprise the full-length alpha Connexin protein. In some aspects, the polypeptide does not comprise the cytoplasmic N-terminal domain of the alpha Connexin. In some aspects, the polypeptide does not comprise the two extracellular domains of the alpha Connexin. In some aspects, the polypeptide does not comprise the four transmembrane domains of the alpha Connexin. In some aspects, the polypeptide does not comprise the cytoplasmic loop domain of the alpha Connexin. In some aspects, the polypeptide does not comprise that part of the sequence of the cytoplasmic carboxyl terminal domain of the alpha Connexin proximal to the fourth transmembrane domain.

The ACT sequence of the polypeptide may be from any alpha Connexin. In some aspects, the alpha Connexin component of the polypeptide can be from a human, murine, bovine, monotrene, marsupial, primate, rodent, cetacean, mammalian, avian, reptilian, amphibian, piscine, chordate, protochordate or other alpha Connexin.

In some aspects, the polypeptide may comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Connexin 43, Cercopithecus Connexin 43, Oryctolagus Connexin 43, Spermophilus Connexin 43, Cricetulus Connexin 43, Phodopus Connexin 43, Rat Connexin 43, Sus Connexin 43, Mesocricetus Connexin 43, Mouse Connexin 43, Cavia Connexin 43, Cow Connexin 43, Erinaceus Connexin 43, Chick Connexin 43, Xenopus Connexin 43, Oryctolagus Connexin 43, Cyprinus Connexin 43, Zebrafish Connexin 43, Danio aequipinnatus Connexin 43, Zebrafish Connexin 43.4, Zebrafish Connexin 44.2, Zebrafish Connexin 44.1, human Connexin45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, Chick Connexin 56, Zebrafish Connexin 39.9, cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin. Amino acid sequences for alpha connexins are known in the art and include those identified in Table 1 by accession number.

TABLE 1
Alpha Connexins

| Protein | Accession No. |
|---|---|
| mouse Connexin 47 | NP_536702 |
| human Connexin 47 | AAH89439 |
| Human Connexin 46.6 | AAB94511 |
| Cow Connexin 46.6 | XP_582393 |
| Mouse Connexin 30.2 | NP_848711 |
| Rat Connexin 30.2 | XP_343966 |
| Human Connexin 31.9 | AAM18801 |
| Dog Connexin 31.9 | XP_548134 |
| Sheep Connexin 44 | AAD56220 |
| Cow Connexin 44 | 146053 |
| Rat Connexin 33 | P28233 |
| Mouse Connexin 33 | AAR28037 |
| Human Connexin 36 | Q9UKL4 |
| mouse Connexin 36 | NP_034420 |
| rat Connexin 36 | NP_062154 |
| dog Connexin 36 | XP_544602 |
| chick Connexin 36 | NP_989913 |
| zebrafish Connexin 36 | NP_919401 |
| morone Connexin 35 | AAC31884 |
| morone Connexin 35 | AAC31885 |
| Cynops Connexin 35 | BAC22077 |
| Tetraodon Connexin 36 | CAG06428 |
| human Connexin 37 | 155593 |
| chimp Connexin 37 | XP_524658 |
| dog Connexin 37 | XP_539602 |
| Cricetulus Connexin 37 | AAR98615 |
| Mouse Connexin 37 | AAH56613 |
| Mesocricetus Connexin 37 | AAS83433 |
| Rat Connexin 37 | AAH86576 |
| mouse Connexin 39 | NP_694726 |
| rat Connexin 39 | AAN17801 |
| human Connexin 40.1 | NP_699199 |
| Xenopus Connexin 38 | AAH73347 |
| Zebrafish Connexin 39.9 | NP_997991 |
| Human Connexin 40 | NP_859054 |
| Chimp Connexin 40 | XP_513754 |
| dog Connexin 40 | XP_540273 |
| cow Connexin 40 | XP_587676 |
| mouse Connexin 40 | AAH53054 |
| rat Connexin 40 | AAH70935 |
| Cricetulus Connexin 40 | AAP37454 |
| Chick Connexin 40 | NP_990835 |
| human Connexin 43 | P17302 |
| Cercopithecus Connexin 43 | AAR33082 |
| Oryctolagus Connexin 43 | AAR33084 |
| Spermophilus Connexin 43 | AAR33086 |
| Cricetulus Connexin 43 | AA061858 |
| Phodopus Connexin 43 | AAR33085 |
| Rat Connexin 43 | AAH81842 |
| Sus Connexin 43 | AAR33087 |
| Mesocricetus Connexin 43 | AA061857 |
| Mouse Connexin 43 | AAH55375 |
| Cavia Connexin 43 | AAU06305 |
| Cow Connexin 43 | NP_776493 |
| Erinaceus Connexin 43 | AAR33083 |
| Chick Connexin 43 | AAA53027 |
| Xenopus-Connexin 43 | NP_988856 |
| Oryctolagus Connexin 43 | AAS89649 |
| Cyprinus Connexin 43 | AAG17938 |
| Zebrafish Connexin 43 | CAH69066 |
| Danio aequipinnatus Connexin 43 | AAC19098 |
| Zebrafish Connexin 43.4 | NP_571144 |
| Zebrafish Connexin 44.2 | AAH45279 |
| Zebrafish Connexin 44.1 | NP_571884 |
| human Connexin 45 | 138430 |
| chimp Connexin 45 | XP_511557 |
| dog Connexin 45 | XP_548059 |
| mouse Connexin 45 | AAH71230 |
| cow Connexin 45 | XP_588395 |
| rat Connexin 45 | AAN17802 |
| chick Connexin 45 | NP_990834 |
| Tetraodon Connexin 45 | CAF93782 |
| chick Connexin 45.6 | 150219 |
| human Connexin 46 | NP_068773 |
| chimp Connexin 46 | XP_522616 |
| mouse Connexin 46 | NP_058671 |
| dog Connexin 46 | XP_543178 |
| rat Connexin 46 | NP_077352 |
| Mesocricetus Connexin 46 | AAS83437 |
| Cricetulus Connexin 46 | AAS77618 |
| Chick Connexin 56 | A45338 |
| Zebrafish Connexin 39.9 | NP_997991 |
| cow Connexin 49 | XP_602360 |
| human Connexin 50 | P48165 |
| chimp Connexin 50 | XP_524857 |
| rat Connexin 50 | NP_703195 |
| mouse Connexin 50 | AAG59880 |
| dog Connexin 50 | XP_540274 |
| sheep Connexin 49 | AAF01367 |
| Mesocricetus Connexin 50 | AAS83438 |
| Cricetulus Connexin 50 | AAR98618 |
| Chick Connexin 50 | BAA05381 |
| human Connexin 59 | AAG09406 |

In some aspects, the compositions disclosed herein comprise a polypeptide which interacts with a domain of a protein that normally mediates the binding of said protein to the carboxy-terminus of an alpha Connexin. In some aspects, the compositions disclosed herein comprise a polypeptide which inhibits the function of a protein that normally binds to alpha Connexin by competitively binding to the protein. For example, nephroblastoma overexpressed protein (NOV) interacts with a Cx43 c-terminal domain (Fu et al., J Biol Chem. 2004 279(35):36943-50). NOV interacts with the carboxy-terminus of alpha Connexins and further interacts with other proteins forming a macromolecular complex. Without being bound by theory, it is thought that the polypeptide of the compositions disclosed herein may inhibit the operation of a molecular machine, such as, for example, one involved in regulating the aggregation of Cx43 gap junction channels by interacting with NOV.

In some aspects, the polypeptide may be flanked by non-alpha Connexin or non-ACT peptide Connexin amino acids. An example of a non-alpha Connexin is the 239 amino acid sequence of enhanced green fluorescent protein. Examples of non-ACT peptide Connexin amino acids include, but are not limited to, the carboxy-terminal 20 to 120 amino acids of human Cx43 (SEQ ID NO: 72); the carboxy-terminal 20 to 120 amino acids of chick Cx43 (SEQ ID NO: 73); the carboxy-terminal 20 to 120 amino acids of human Cx45 (SEQ ID NO: 74); the carboxy-terminal 20 to 120 amino acids of chick Cx45 (SEQ ID NO: 75); the carboxy-terminal 20 to 120 amino of human Cx37 (SEQ ID NO: 76); and the carboxy-terminal 20 to 120 amino acids of rat Cx33 (SEQ ID NO: 77).

The carboxy-terminal-most amino acid sequences of alpha Connexins are characterized by multiple distinctive and conserved features (see Table 2). This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation.

In some aspects, the polypeptide of the compositions disclosed herein comprises a type II PDZ binding motif (Φ-x-Φ; wherein x=any amino acid and Φ=a Hydrophobic amino acid; e.g., Table 2, BOLD). PDZ motifs are consensus sequences that are normally, but not always, located at the extreme intracellular carboxyl terminus. Four types of PDZ motifs have been classified: type I (S/T-x-Φ), type II (Φ-x-Φ), type III (Ψ-x-Φ) and type IV (D-x-V), where x is any amino acid, Φ is a hydrophobic residue (V, I, L, A, G, W, C, M, F) and Ψ is a basic, hydrophilic residue (H, R, K). (Songyang, Z., et al. 1997. Science 275, 73-77).

In some aspects, the polypeptide of the compositions disclosed herein may inhibit the binding of an alpha Connexin to a protein comprising a PDZ domain. PDZ domains were originally identified as conserved sequence elements within the postsynaptic density protein PSD95/SAP90, the Drosophila tumor suppressor dlg-A, and the tight junction protein ZO-1. Although originally referred to as GLGF or DHR motifs, they are now known by an acronym representing these first three PDZ containing proteins (PSD95/DLG/ZO-1). These 80-90 amino acid sequences have now been identified in well over 75 proteins and are characteristically expressed in multiple copies within a single protein. The PDZ domain is a specific type of protein-interaction module that has a structurally well-defined interaction ‘pocket’ that can be filled by a PDZ-binding motif.

In some aspects, the polypeptide of the disclosed compositions comprises, proximal to the PDZ binding motif—Proline (P) and/or Glycine (G) hinge residues; a high frequency phospho-Serine (S) and/or phospho-Threonine (T) residues; and a high frequency of positively charged Arginine (R), Lysine (K) and negatively charged Aspartic acid (D) or Glutamic acid (E) amino acids. In some aspects, the P and G residues occur in clustered motifs (e.g., Table 2, italicized) proximal to the carboxy-terminal type II PDZ binding motif. The S and T phosphor-amino acids, in some aspects, are organized in clustered, repeat-like motifs (e.g., Table 2, underlined). ACT peptide organization of Cx43 is highly conserved from humans to fish (e.g., compare Cx43 ACT sequences for humans and zebrafish in Table 2). Further, the ACT peptide organization of Cx45 is highly conserved from humans to birds (e.g., compare Cx45 ACT sequences for humans and chick in Table 2). The ACT peptide organization of Cx36 is also highly conserved from primates to fish (e.g., compare Cx36 ACT sequences for chimp and zebrafish in Table 2).

TABLE 2
Alpha Connexin Carboxy-Terminal (ACT) Amino Acid Sequences

| Gene | Sequence | SEQ ID NO |
|---|---|---|
| Human alpha Cx43 | P SSRA SSR PRP D DLEI | (SEQ ID NO: 9) |
| Chick alpha Cx43 | P S RA SSRA SSR PRP D DLEI | (SEQ ID NO: 29) |
| Zebrafish alpha Cx43 | P CSRA SSRM SSRA R P D DLDV | (SEQ ID NO: 90) |
| Human alpha Cx45 | G SNKS TA SSKS GDG KN SVWI | (SEQ ID NO: 30) |
| Chick alpha Cx45 | G SNKSS A SSKS GDG KN SVWI | (SEQ ID NO: 31) |
| Human alpha Cx46 | G RA SKAS RASS [symbol] RAR [symbol] E DLAI | SEQ ID NO: 32) |
| Human alpha Cx46.6 | G SASS RD [symbol] K TVWI | (SEQ ID NO: 33) |
| Chimp alpha Cx36 | P RVSV PNFG R TQ SSD SAYV | (SEQ ID NO: 34) |
| Chick alpha Cx36 | P RMSM PNFG R TQ SSD S AYV | (SEQ ID NO: 35) |
| Zebrafish alpha Cx36 | P RMSM PNFG R TQ SSD S AYV | (SEQ ID NO: 91) |
| Human alpha Cx47 | P RAGSEK G SASS R DG KT TVWI | (SEQ LD NO: 36) |
| Human alpha Cx40 | G HRL [symbol] H [symbol] YHSDKRRL SKASS | (SEQ ID NO: 37) |
| | KARSD DLSV | |
| Human alpha Cx50 | P ELTTDDAR P LSRL SKASS RARSD | (SEQ ID NO: 38) |
| | DLTV | |
| Human alpha Cx59 | P NHVV SLTN NLI GRRVP T DLQI | (SEQ ID NO: 39) |
| Rat alpha Cx33 | P S CV SSS A VLTTIC SS DQVV PVG | (SEQ ID NO: 40) |
| | L SS FYM | |
| Sheep alpha Cx44 | G R SSKA SKSS GG RARAA DLAI | (SEQ ID NO: 41) |
| Human beta Cx26 | LC YLLIR YCSGK SKKPV | (SEQ ID NO: 42) |

In some aspects, there is a conserved proline or glycine residue positioned about 17 to 30 amino acids from the carboxyl terminus of the polypeptide (Table 2). Non-limiting examples are as follows. In human Cx43 a proline residue at amino acid 363 is positioned 19 amino acids from the carboxyl terminus. In chick Cx43 a proline residue at amino acid 362 is positioned 18 amino acids from the carboxyl terminus. In human Cx45 a glycine residue at amino acid 377 is positioned 19 amino acids from the carboxyl terminus. In rat Cx33, a proline residue at amino acid 258 is positioned 28 amino acids back from the carboxyl terminus. In some aspects, the polypeptide of the compositions disclosed herein do not comprise amino acids proximal to this conserved proline or glycine residue positioned about 17 to 30 amino acids from the carboxyl terminus.

In some aspects, the polypeptide of the compositions disclosed herein comprises one, two, three or all of the amino acid motifs selected from the group consisting of 1) a type II PDZ binding motif, 2) Proline (P) and/or Glycine (G) hinge residues; 3) clusters of phospho-Serine (S) and/or phospho-Threonine (T) residues; and 4) a high frequency of positively charged Arginine (R) and Lysine (K) and negatively charged Aspartic acid (D) and/or Glutamic acid (E) amino acids). In some aspects, the polypeptide comprises a type II PDZ binding motif at the carboxy-terminus, Proline (P) and/or Glycine (G) hinge residues proximal to the PDZ binding motif, and positively charged residues (K, R, D, E) proximal to the hinge residues.

In some aspects, greater than about 50%, greater than about 60%, greater than about 70%, greater than about 80%, or greater than about 90% of the amino acids of the polypeptide is comprised of one or more of Proline (P), Glycine (G), phospho-Serine (S), phospho-Threonine (T), Arginine (R), Lysine (K), Aspartic acid (D), or Glutamic acid (E) amino acid residues. The amino acids Proline (P), Glycine (G), Arginine (R), Lysine (K), Aspartic acid (D), and Glutamic acid (E) may be important for determining protein structure and function. Without being bound by theory, it is thought that Proline and Glycine residues provide for tight turns in the 3D structure of proteins, enabling the generation of folded conformations of the polypeptide required for function. Charged amino acid sequences are often located at the surface of folded proteins and may be important for chemical interactions mediated by the polypeptide including protein-protein interactions, protein-lipid interactions, enzyme-substrate interactions and protein-nucleic acid interactions. In some aspects, the polypeptide of the disclosed compositions comprises Proline (P) and Glycine (G) Lysine (K), Aspartic acid (D), and/or Glutamic acid (E) rich regions proximal to the type II PDZ binding motif.

The 18 carboxy-terminal-most amino acid sequence of alpha Cx37 represents an exceptional variation on the ACT peptide theme. The Cx37 ACT-like sequence is GQKPPSRPSSSASKKQ*YV (SEQ ID NO: 43). Thus the carboxy terminal 4 amino acids of Cx37 conform only in part to a type II PDZ binding domain. Instead of a classical type II PDZ binding domain, Cx37 has a neutral Q* at position 2 where a hydrophobic amino acid would be expected. As such Cx37 comprises what might be termed a type II PDZ binding domain-like sequence. Nonetheless, Cx37 strictly maintains all other aspects of ACT peptide organization including clustered serine residues, frequent R and K residues and a P-rich sequence proximal to the PDZ binding domain-like sequence; and therefore, shares an overall level of conservation of ACT-like organization with the other >70 alpha Connexins listed above. Further, the functional properties of Cx37 ACT-like carboxy terminus are also shared with the other alpha Connexins.

For comparison, the beta Connexin Cx26 is shown in Table 2. Cx26 has no carboxyl terminal type II PDZ binding motif; less than 30% of the carboxyl terminal most amino acids comprise S, T, R, D or E residues; it has no evidence of motifs proximal to a type II PDZ binding motif or PDZ binding like motif containing clusters of P and G hinge residues; and no evidence of clustered, repeat-like motifs of serine and threonine phospho-amino acids. Cx26 does have three Lysine (K) residues, clustered one after the other near the carboxy terminus of the sequence. However, no alpha Connexin surveyed in the >70 alpha Connexins listed above was found to display this feature of three repeated K residues domain at carboxy terminus (Cx26 is a beta connexin, thus by definition does not have an ACT domain).

In some aspects, polypeptides of the compositions disclosed herein comprise variants, derivatives, and fragments of the polypeptides. In some aspects, the variants of the polypeptides comprise amino acid modifications. For example, amino acid sequence modifications may include, but are not limited to, amino acid substitutions, amino acid insertions and amino acid deletions. In preferred aspects, the polypeptides comprise conservative substitutions, which refers to the replacement of one amino acid residue with another that is biologically and/or chemically similar. In some aspects, the conservative substitutions do not appreciably alter the structure or function of the polypeptides. Exemplary conservative substitutions are listed in Table 3. In some aspects, polypeptides may comprise any combination of substitutions, deletions, insertions or other amino acid substitutions.

TABLE 3
Amino Acid Substitution

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

In some aspects, the conservative substitutions are generated by standard procedures such as site-directed mutagenesis or PCR; standard peptide synthesis methods; and alanine scan. Conservative substitutions are described in further detail in Ben-Bassat et al., (J. Bacterial. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology. In some aspects, the biological activity of the polypeptide is decreased by not more than 25%, not more than 20%, not more that 15%, not more than 10% or not more than 5% when the polypeptide has one or more conservative substitutions. In some aspects, the polypeptides comprise one or more amino acid substitutions. In some aspects, the polypeptides comprise 2-10 conservative substitutions, 4-8 conservative substitutions, 5-7 conservative substitutions, such as, for example 2, 3, 4, 5, 6, 7, 8, 9 or 10 conservative substitutions.

In particular aspects, the polypeptide comprises the amino acid sequence of SEQ ID NO: 1. In some aspects, the polypeptide may comprise the amino acid sequence of SEQ ID NO: 3, which comprises a single conservative substitution within the sequence SEQ ID NO: 1. In some aspects, the polypeptide may comprise the amino acid sequence of SEQ ID NO: 4, which comprises three conservative substitutions within the sequence SEQ ID NO: 1.

TABLE 4
ACT Polypeptide Variants

| Sequence | SEQ ID NO |
|---|---|
| RPRPDDLEI | SEQ ID NO: 1 |
| RPRPDDLEV | SEQ ID NO: 3 |
| RPRPDDVPV | SEQ ID NO: 4 |
| SSRASSRASSRPRPDDLEV | SEQ ID NO: 44 |
| RPKPDDLEI | SEQ ID NO: 45 |
| SSRASSRASSRPKPDDLEI | SEQ ID NO: 46 |
| RPKPDDLDI | SEQ ID NO: 47 |
| SSRASSRASSRPRPDDLDI | SEQ ID NO: 48 |
| SSRASTRASSRPRPDDLEI | SEQ ID NO: 49 |
| RPRPEDLEI | SEQ ID NO: 50 |
| SSRASSRASSRPRPEDLEI | SEQ ID NO: 51 |
| GDGKNSVWV | SEQ ID NO: 52 |
| SKAGSNKSTASSKSGDGKNSVWV | SEQ ID NO: 53 |
| GQKPPSRPSSSASKKLYV | SEQ ID NO: 54 |

In some aspects, the polypeptides of the disclosed compositions may comprise at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to the amino acid sequence of a defined c-terminus of an alpha Connexin (ACT). An exemplary polypeptide (SEQ ID NO: 4) has about 66% sequence identity to the same stretch of 9 amino acids occurring on the carboxy-terminus of human Cx43 (SEQ ID NO: 1).

In some aspects, the polypeptides of the disclosed compositions may comprise at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to SEQ ID NO:1, SEQ ID NO: 2, SEQ ID NO: 9, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:90 or ID NO:91.

In some aspects, the polypeptide may comprise the amino acid sequence SEQ ID NO: 1, SEQ ID NO: 2, SEQ ID NO:9, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:90 or ID NO:91 or conservative variants or fragments thereof. In some aspects, the polypeptide may consist of the amino acid sequence SEQ ID NO: 1, SEQ ID NO: 2, SEQ ID NO: 9, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:90 or ID NO:91 or conservative variants or fragments thereof.

In some aspects, the polypeptides of the disclosed compositions comprise Serine (S) and/or Threonine (T) rich sequences or motifs. In some aspects, the serine and/or threonine in the polypeptides is phosphorylated. Without being bound by theory, it is thought that the phosphorylated serine and/or threonine rich sequences may modify the function of ACT peptides by increasing or decreasing functional efficacy of the polypeptides.

In some aspects, N-glycosylation (Asn-X-Thr/Ser) or O-glycosylation (Ser or Thr) sites may be inserted in the polypeptide. In some aspects, cysteine or other labile residues in the polypeptide may be deleted. In some aspects, potential proteolysis sites, e.g. Arg, may be deleted or substituted; for example, a basic residue may be deleted or substituted with a glutaminyl or histidyl residue. In some aspects, the glutaminyl and asparaginyl residues of the polypeptide are post-translationally deamidated to the corresponding glutamyl and asparyl residues. Other post-translational modifications include, but are not limited to, hydroxylation of proline and lysine; methylation of the o-amino groups of lysine, arginine, and histidine side chains (T. E. Creighton, Proteins: Structure and Molecular Properties, W. H. Freeman & Co., San Francisco pp 79-86 [1983]); acetylation of the N-terminal amine; and amidation of the C-terminal carboxyl.

In some aspects, amino acid and peptide analogs may be incorporated into the polypeptides of the disclosed compositions. In some aspects, the polypeptides may comprise D-amino acids or amino acids which have a different functional substituent than the amino acids shown in Table 3. Amino acid analogs may be incorporated into polypeptide chains using standard techniques, such as charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs). Further, the polypeptides may comprise opposite stereoisomers of naturally occurring peptides or stereoisomers of peptide analogs.

In some aspects, the polypeptides may comprise linkages which are not natural peptide linkages. For example, linkages for amino acids or amino acid analogs can include CH₂NH—, —CH₂S—, —CH₂—CH₂—, —CH═CH— (cis and trans), COCH₂—, —CH(OH)CH₂, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J Chem. Soc Perkin Trans. I 307-314 (1982) (—CH—CH—, cis and trans); Almquist et al. J Med. Chem. 23:1392-1398 (1980)

(—COCH₂—); Jennings-White et al. Tetrahedron Lett 23:2533 (1982) (—COCH₂—); Szelke et al. European Appln, EP 45665 CA (1982): 97:39405 (1982) (—CH(OH) CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. In some aspects, the peptide analogs can have more than one atom between the bond atoms, such as b-alanine, g-aminobutyric acid, and the like.

Without being bound by theory, it is thought that amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others. D-amino acids may be used to generate more stable peptides, because D amino acids are not recognized by peptidases and such. Systematic substitution of one or more amino acids of a consensus sequence with a D-amino acid of the same type (e.g., D-lysine in place of L-lysine) may be used to generate more stable peptides. Cysteine residues can be used to cyclize or attach two or more peptides together. Without being bound by theory, it is thought that this can be beneficial to constrain peptides into particular conformations. (Rizo and Gierasch Ann. Rev. Biochem. 61:387 (1992), incorporated herein by reference).

In some aspects, the polypeptides of the disclosed compositions may comprise a cellular internalization transporter or sequence. The cellular internalization sequence can be any internalization sequence known or newly discovered in the art, or conservative variants thereof. Without being bound by theory, it is thought that the efficiency of cytoplasmic localization of the polypeptide is enhanced by cellular internalization transporter chemically linked in cis or trans with the polypeptide. In some aspects, the polypeptide may be transduced into cells in combination with Tat-HA peptide or exposure to light. Without being bound by theory, it is thought that the efficiency of cell internalization transporters is enhanced further by light or co-transduction of cells with Tat-HA peptide.

Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol) (see Table 5).

TABLE 5
Cell Internalization Transporters

| Name | Sequence | SEQ ID NO |
|---|---|---|
| Antp | RQPKIWFPNRRKPWKK | (SEQ ID NO: 7) |
| HIV-Tat | GRKKRRQRPPQ | (SEQ ID NO: 14) |
| Penetratin | RQIKIWFQNRRMKWKK | (SEQ ID NO: 15) |
| Antp-3A | RQIAIWFQNRRMKWAA | (SEQ ID NO: 16) |
| Tat | RKKRRQRRR | (SEQ ID NO: 17) |
| Buforin II | TRSSRAGLQFPVGRVHRLLRK | (SEQ ID NO: 18) |
| Transportan | GWTLNSAGYLLGKINKALAALAKKIL | (SEQ ID NO: 19) |
| model amphipathic | KLALKLALKALKAALKLA | (SEQ ID NO: 20) |
| peptide (MAP | | |
| K-FGF | AAVALLPAVLLALLAP | (SEQ ID NO: 21) |
| Ku70 | VPMLK-PMLKE | (SEQ ID NO: 22) |
| Prion | MANLGYWLLALFVTMWTDVGLCKKRPKP | (SEQ ID NO: 23) |
| pVEC | LLIILARRIRKQAHAHSK | (SEQ ID NO: 24) |
| Pep-1 | KETWWWETWWTEWSQPKKKRKV | (SEQ ID NO: 25) |
| SynB1 | RGGRLSSYSRRRFSTSTGR | (SEQ ID NO: 26) |
| Pep-7 | SDLWEMMMVSLACQY | (SEQ ID NO: 27) |
| HN-1 | TSPLNIHNGQKL | (SEQ ID NO: 28) |
| BGSC (Bis- Guanidinium- Spermidine- Cholesterol) | 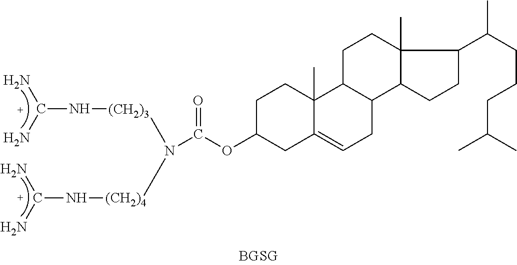 | |
| BGTC (Bis- Guanidinium-Tren- Cholesterol) | 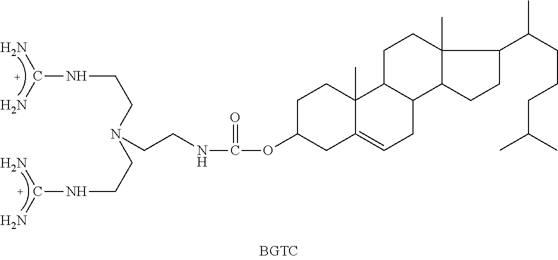 [SMILES: CC(C)CCCC(C)C1CCC2C3CC=C4CC(OC(=O)NCCN(CCNC(N)N)CCNC(N)N)CCC4(C)C3CCC12C] | |

The polypeptides of the disclosed compositions may further comprise the amino acid sequence represented by SEQ ID NO: 7, SEQ ID NO: 14 (Bucci, M. et al. 2000. Nat. Med. 6, 1362-1367), SEQ ID NO: 15 (Derossi, D., et al. 1994. Biol. Chem. 269, 10444-10450), SEQ ID NO: 16 (Fischer, P. M. et al. 2000. J. Pept. Res. 55, 163-172), SEQ ID NO: 17 (Frankel, A. D. & Pabo, C. O. 1988. Cell 55, 1189-1193; Green, M. & Loewenstein, P. M. 1988. Cell 55, 1179-1188), SEQ ID NO: 18 (Park, C. B., et al. 2000. Proc. Natl Acad. Sci. USA 97, 8245-8250), SEQ ID NO: 19 (Pooga, M., et al. 1998. FASEB J. 12, 67-77), SEQ ID NO: 20 (Oehlke, J. et al. 1998. Biochim. Biophys. Acta. 1414, 127-139), SEQ ID NO: 21 (Lin, Y Z., et al. 1995. J Biol. Chem. 270, 14255-14258), SEQ ID NO: 22 (Sawada, M., et al. 2003. Nature Cell Biol. 5, 352-357), SEQ ID NO: 23 (Lundberg, P. et al. 2002. Biochem. Biophys. Res. Commun. 299, 85-90), SEQ ID NO: 24 (Elmquist, A., et al. 2001. Exp. Cell Res. 269, 237-244), SEQ ID NO: 25 (Morris, M. C., et al. 2001. Nature Biotechnol. 19, 1173-1176), SEQ ID NO:26 (Rousselle, C. et al. 2000. Mol. Pharmacol. 57, 679-686), SEQ ID NO: 27 (Gao, C. et al. 2002. Bioorg. Med. Chem. 10, 4057-4065), or SEQ ID NO: 28 (Hong, F. D. & Clayman, G. L. 2000. Cancer Res. 60, 6551-6556). The polypeptide of the disclosed compositions may further comprise BGSC (Bis-Guanidinium-Spermidine-Cholesterol) or BGTC (Bis-Guanidinium-Tren-Cholesterol) (Vigneron, J. P. et al. 1998. Proc. Natl. Acad. Sci. USA. 93, 9682-9686). The preceding references are hereby incorporated herein by reference in their entirety for the teachings of cellular internalization vectors and sequences. Any other internalization sequences now known or later identified may be combined with any of the polypeptide of the compositions disclosed herein.

The polypeptides of the disclosed compositions may comprise any ACT sequence in combination with any cell internalization sequence. Non-limiting examples of said combinations are given in Table 6. The polypeptide of the disclosed compositions may comprise an Antennapedia sequence comprising amino acid sequence SEQ ID NO: 7; an amino acid sequence SEQ ID NO: 2, SEQ ID NO: 8, SEQ ID NO: 10, SEQ ID NO: 11, or SEQ ID NO: 12. In particular embodiments, the polypeptide comprises the 9 carboxy-terminal most amino acids of connexin 43 linked to an Antennapedia sequence, and comprises an amino acid sequence of SEQ ID NO: 2

TABLE 6
Exemplary polypeptides

| Sequence | SEQ ID NO |
|---|---|
| RQPKIWFPNRRKPWKK PSSRASSRASSRPRPDDLEI | SEQ ID NO: 8 |
| RQPKIWFPNRRKPWKK RPRPDDLEI | SEQ ID NO: 2 |
| RQPKIWFPNRRKPWKK RPRPDDLEV | SEQ ID NO: 10 |
| RQPKIWFPNRRKPWKK RPRPDDVPV | SEQ ID NO: 11 |
| RQPKIWFPNRRKPWKK KARSDDLSV | SEQ ID NO: 12 |
| GRKKRRQRPPQ RPRPDDLEI | SEQ ID NO: 56 |
| RQIKIWFQNRRMKWKK RPRPDDLEI | SEQ ID NO: 57 |
| RQIAIWFQNRRMKWAA RPRPDDLEI | SEQ ID NO: 58 |
| RKKRRQRRR RPRPDDLEI | SEQ ID NO: 59 |
| TRSSRAGLQFPVGRVHRLLRK RPRPDDLEI | SEQ ID NO: 60 |
| GWTLNSAGYLLGKINKALAAL AKKIL RPRPDDLEI | SEQ ID NO: 61 |
| KLALKLALKALKAALKLA RPRPDDLEI | SEQ ID NO: 62 |
| AAVALLPAVLLALLAP RPRPDDLEI | SEQ ID NO: 63 |
| VPMLKPMLKE RPRPDDLEI | SEQ ID NO: 64 |
| MANLGYWLLALFVTMWTDVG LCKKRPKP RPRPDDLEI | SEQ ID NO: 65 |
| LLIILRRRIRKQAHAHSK RPRPDDLEI | SEQ ID NO: 66 |
| KETWWETWWTEWSQPKKKRKV RPRPDDLEI | SEQ ID NO: 67 |
| RGGRLSYSRRRFSTSTGR RPRPDDLEI | SEQ ID NO: 68 |
| SDLWEMMMVSLACQY RPRPDDLEI | SEQ ID NO: 69 |
| TSPLNIHNGQKL RPRPDDLEI | SEQ ID NO: 70 |

The term “αCT1” is used interchangeably herein with “aCT1” or “ACT1”, and refers to the 25 amino acid polypeptide according to SEQ ID NO: 2.

Further details about the polypeptides disclosed herein are described in U.S. Pat. No. 7,786,074, which is incorporated herein by reference in its entirety.

### Nanoparticle Compositions

In some embodiments, the present disclosure provides a composition comprising one or more nanoparticles, wherein the nanoparticles comprise one or more biodegradable or biocompatible polymers and a therapeutically effective amount of an alpha connexin polypeptide provided herein. In some embodiments, the present disclosure provides a composition comprising one or more nanoparticles, wherein the nanoparticles comprise one or more biodegradable or biocompatible polymers and a therapeutically effective amount of a peptide comprising an amino acid sequence according to SEQ ID NO: 1. In some embodiments, the peptide further comprises a cellular internalization sequence. The cellular internalization sequence may comprise an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol). In some embodiments, the peptide comprises an amino acid sequence according to SEQ ID NO: 2.

The nanoparticles of the present disclosure may comprise one or more biodegradable or biocompatible polymers. As used herein the term “biodegradable or biocompatible polymers” refer to polymers that are biodegradable or biocompatible, or both biodegradable and biocompatible. Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise taken up by such cells. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible. For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

Suitable biodegradable or biocompatible polymers will be readily apparent to the skilled artisan. For example, the one or more biodegradable or biocompatible polymers of the present disclosure include, but are not limited to poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyethylene, polypropylene, poly(ethylene glycol) (PEG), polyalkylene oxides (PEO), poly(ethylene terephthalate), polyvinyl alcohols (PVA), polyvinyl ethers, poly(vinyl acetate), poly(vinyl chloride) (PVC), polyvinylpyrrolidone (PVP), polysiloxanes, polystyrene, polyurethanes, alkyl celluloses, hydroxyalkyl celluloses, cellulose ethers, cellulose esters, nitro celluloses, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate), polydioxanone, polydioxanone copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol. In some embodiments, the one or more biodegradable or biocompatible polymers are PLGA. In some embodiments, the one or more biodegradable or biocompatible polymers are PLGA and PVA.

In some embodiments, the PLGA has a Mw from about 4,000 to about 240,000 Da. In some embodiments, the PLGA has a Mw from about 7,000 to about 17,000 Da. For example, the PLGA may have a Mw of about 7,000 Da, about 8,000 Da, about 9,000 Da, about 10,000 Da, about 11,000 Da, about 12,000 Da, about 13,000 Da, about 14,000 Da, about 15,000 Da, about 16,000 Da, or about 17,000 Da, including all integers and ranges therebetween. In some embodiments, the PLGA is 50:50 lactic acid:glycolic acid, acid terminated. In some embodiments, the PLGA is 5:95 lactic acid:glycolic acid, acid terminated. In some embodiments, the PLGA is 10:90 lactic acid:glycolic acid, acid terminated. In some embodiments, the PLGA is 15:85 lactic acid:glycolic acid, acid terminated. In some embodiments, the PLGA is 20:80 lactic acid:glycolic acid, acid terminated. In some embodiments, the PLGA is 25:75 lactic acid:glycolic acid, acid terminated. In some embodiments, the PLGA is 30:70 lactic acid:glycolic acid, acid terminated. In some embodiments, the PLGA is 35:65 lactic acid:glycolic acid, acid terminated. In some embodiments, the PLGA is 40:60 lactic acid:glycolic acid, acid terminated. In some embodiments, the PLGA is 45:55 lactic acid:glycolic acid, acid terminated. In some embodiments, the PLGA is 50:50 lactic acid:glycolic acid, acid terminated. In some embodiments, the PLGA is 55:45 lactic acid:glycolic acid, acid terminated. In some embodiments, the PLGA is 60:40 lactic acid:glycolic acid, acid terminated. In some embodiments, the PLGA is 65:35 lactic acid:glycolic acid, acid terminated. In some embodiments, the PLGA is 70:30 lactic acid:glycolic acid, acid terminated. In some embodiments, the PLGA is 75:25 lactic acid:glycolic acid, acid terminated. In some embodiments, the PLGA is 80:20 lactic acid:glycolic acid, acid terminated. In some embodiments, the PLGA is 85:15 lactic acid:glycolic acid, acid terminated. In some embodiments, the PLGA is 90:10 lactic acid:glycolic acid, acid terminated. In some embodiments, the PLGA is 95:5 lactic acid:glycolic acid, acid terminated.

In some embodiments, the PVA has a Mw from about 8,000 to about 186,000 Da, for example from about 13,000 to about 23,000 Da. For example, the PVA may have a Mw of about 13,000 Da, about 14,000 Da, about 15,000 Da, about 16,000 Da, about 17,000 Da, about 18,000 Da, about 19,000 Da, about 20,000 Da, about 21,000 Da, about 22,000 Da, or about 23,000 Da, including all integers and ranges therebetween.

In some embodiments, the amount of PVA is between about 0.05% (w/v) to about 5% (w/v). For example, the amount of PVA in the composition is about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), about 1.2% (w/v), about 1.3% (w/v), about 1.4% (w/v), about 1.5% (w/v), about 1.6% (w/v), about 1.7% (w/v), about 1.8% (w/v), about 1.9% (w/v), about 2.0% (w/v), 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), about 3.0% (w/v), 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4.0% (w/v), 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), or about 5.0% (w/v), including all integers and ranges therebetween. In some embodiments, the amount of PVA may range between about 0.1% (w/v) to about 5% (w/v). For example, the amount of PVA may range between about 0.1% (w/v) to about 5% (w/v), between about 0.1% (w/v) to about 4% (w/v), between about 0.1% (w/v) to about 3% (w/v), between about 0.1% (w/v) to about 2% (w/v), between about 0.1% (w/v) to about 1% (w/v), between about 0.2% (w/v) to about 5% (w/v), between about 0.2% (w/v) to about 4% (w/v), between about 0.2% (w/v) to about 3% (w/v), between about 0.2% (w/v) to about 2% (w/v), between about 0.2% (w/v) to about 1% (w/v), between about 0.3% (w/v) to about 5% (w/v), between about 0.3% (w/v) to about 4% (w/v), between about 0.3% (w/v) to about 3% (w/v), between about 0.3% (w/v) to about 2% (w/v), between about 0.3% (w/v) to about 1% (w/v), between about 0.4% (w/v) to about 5% (w/v), between about 0.4% (w/v) to about 4% (w/v), between about 0.4% (w/v) to about 3% (w/v), between about 0.4% (w/v) to about 2% (w/v), between about 0.4% (w/v) to about 1% (w/v), between about 0.5% (w/v) to about 5% (w/v), between about 0.5% (w/v) to about 4% (w/v), between about 0.5% (w/v) to about 3% (w/v), between about 0.5% (w/v) to about 3% (w/v), between about 0.5 (w/v) to about 2% (w/v), between about 0.5% (w/v) to about 1% (w/v), between about 1% (w/v) to about 5% (w/v), between about 1% (w/v) to about 5% (w/v), between about 1% (w/v) to about 4% (w/v), between about 1% (w/v) to about 3% (w/v), between about 1% (w/v) to about 2% (w/v), between about 2% (w/v) to about 5% (w/v), between about 2% (w/v) to about 4% (w/v), between about 2% (w/v) to about 3% (w/v), between about 3% (w/v) to about 5% (w/v), or between about 4% (w/v) to about 5% (w/v). In some embodiments, the amount of PVA is between about 0.3% (w/v) to about 2.5% (w/v).

In some embodiments, the amount of PLGA is between about 2% (w/v) to about 10% (w/v). For example, the amount of PLGA in the composition is about 2.0% (w/v), 2.1% (w/v), about 2.2% (w/v), about 2.3% (w/v), about 2.4% (w/v), about 2.5% (w/v), about 2.6% (w/v), about 2.7% (w/v), about 2.8% (w/v), about 2.9% (w/v), about 3.0% (w/v), 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4.0% (w/v), 4.1% (w/v), about 4.2% (w/v), about 4.3% (w/v), about 4.4% (w/v), about 4.5% (w/v), about 4.6% (w/v), about 4.7% (w/v), about 4.8% (w/v), about 4.9% (w/v), about 5.0% (w/v), about 5.1% (w/v), about 5.2% (w/v), about 5.3% (w/v), about 5.4% (w/v), about 5.5% (w/v), about 5.6% (w/v), about 5.7% (w/v), about 5.8% (w/v), about 5.9% (w/v), about 6.0% (w/v), 6.1% (w/v), about 6.2% (w/v), about 6.3% (w/v), about 6.4% (w/v), about 6.5% (w/v), about 6.6% (w/v), about 6.7% (w/v), about 6.8% (w/v), about 6.9% (w/v), about 7.0% (w/v), 7.1% (w/v), about 7.2% (w/v), about 7.3% (w/v), about 7.4% (w/v), about 7.5% (w/v), about 7.6% (w/v), about 7.7% (w/v), about 7.8% (w/v), about 7.9% (w/v), about 8.0% (w/v), 8.1% (w/v), about 8.2% (w/v), about 8.3% (w/v), about 8.4% (w/v), about 8.5% (w/v), about 8.6% (w/v), about 8.7% (w/v), about 8.8% (w/v), about 8.9% (w/v), about 9.0% (w/v), about 9.1% (w/v), about 9.2% (w/v), about 9.3% (w/v), about 9.4% (w/v), about 9.5% (w/v), about 9.6% (w/v), about 9.7% (w/v), about 9.8% (w/v), about 9.9% (w/v), or about 10.0% (w/v), including all integers and ranges therebetween. In some embodiments, the amount of PLGA may range between about 2% (w/v) to about 9% (w/v), between about 2% (w/v) to about 7% (w/v), between about 2% (w/v) to about 5% (w/v), between about 2% (w/v) to about 2% (w/v), between about 3% (w/v) to about 10% (w/v), between about 3% (w/v) to about 9% (w/v), between about 3% (w/v) to about 7% (w/v), between about 3% (w/v) to about 5% (w/v), between about 4% (w/v) to about 10% (w/v), between about 4% (w/v) to about 8% (w/v), between about 4% (w/v) to about 6% (w/v), between about 5% (w/v) to about 10% (w/v), between about 5% (w/v) to about 8% (w/v), between about 5% (w/v) to about 7% (w/v), between about 6% (w/v) to about 10% (w/v), between about 6% (w/v) to about 9% (w/v), between about 6% (w/v) to about 8% (w/v), between about 7% (w/v) to about 10% (w/v), between about 7% (w/v) to about 9% (w/v), or between about 8% (w/v) to about 10% (w/v).

In some embodiments, the average diameter of the nanoparticles is between about 10 nm and about 1000 nm. For example, the average diameter of the nanoparticle may be about 10 nm, about 20 nm, about 30 nm, about 40 nm, about 50 nm, about 60 nm, about 70 nm, about 80 nm, about 90 nm, about 100 nm, 110 nm, about 120 nm, about 130 nm, about 140 nm, about 150 nm, about 160 nm, about 170 nm, about 180 nm, about 190 nm, about 200 nm, 210 nm, about 220 nm, about 230 nm, about 240 nm, about 250 nm, about 260 nm, about 270 nm, about 280 nm, about 290 nm, about 300 nm, 310 nm, about 320 nm, about 330 nm, about 340 nm, about 350 nm, about 360 nm, about 370 nm, about 380 nm, about 390 nm, about 400 nm, 410 nm, about 420 nm, about 430 nm, about 440 nm, about 450 nm, about 460 nm, about 470 nm, about 480 nm, about 490 nm, about 500 nm, 510 nm, about 520 nm, about 530 nm, about 540 nm, about 550 nm, about 560 nm, about 570 nm, about 580 nm, about 590 nm, about 600 nm, 610 nm, about 620 nm, about 630 nm, about 640 nm, about 650 nm, about 660 nm, about 670 nm, about 680 nm, about 690 nm, about 700 nm, 710 nm, about 720 nm, about 730 nm, about 740 nm, about 750 nm, about 760 nm, about 770 nm, about 780 nm, about 790 nm, about 800 nm, 810 nm, about 820 nm, about 830 nm, about 840 nm, about 850 nm, about 860 nm, about 870 nm, about 880 nm, about 890 nm, about 900 nm, 910 nm, about 920 nm, about 930 nm, about 940 nm, about 950 nm, about 960 nm, about 970 nm, about 980 nm, about 990 nm, or about 1000 nm, including all integers and ranges therebetween. In some embodiments, the average diameter of the nanoparticles ranges from about 10 nm to about 1000 nm. For example, the average diameter of the nanoparticles ranges from about 10 nm to about 1000 nm, about 10 nm to about 800 nm, about 10 nm to about 600 nm, about 10 nm to about 400 nm, about 10 nm to about 200 nm, about 10 nm to about 50 nm, about 30 nm to about 1000 nm, about 30 nm to about 800 nm, about 30 nm to about 600 nm, about 30 nm to about 500 nm, about 30 nm to about 400 nm, about 30 nm to about 200 nm, about 30 nm to about 50 nm, about 50 nm to about 1000 nm, about 50 nm to about 800 nm, about 50 nm to about 600 nm, about 50 nm to about 400 nm, about 50 nm to about 200 nm, about 50 nm to about 100 nm, about 100 nm to about 1000 nm, about 100 nm to about 800 nm, about 100 nm to about 600 nm, about 100 nm to about 400 nm, about 100 nm to about 300 nm, about 100 nm to about 200 nm, or about 150 nm to about 200 nm. In some embodiments, the average diameter of the nanoparticles is between about 30 nm and about 500 nm. In some embodiments, the average diameter of the nanoparticles is between about 100 nm and about 300 nm. In some embodiments, the average diameter of the nanoparticles is between about 100 nm and about 200 nm. In some embodiments, the average diameter of the nanoparticles is about 180 nm.

The nanoparticles of the present disclosure have a uniform particle size and little to no agglomeration. Uniform particle size and little to no agglomeration is desirable because it eliminates the need for costly processing steps such as wet and/or dry milling steps. For example, in some embodiments, the average diameter of the nanoparticles is between about 100 nm and about 300 nm. In some embodiments, the average diameter of the nanoparticles is between about 100 nm and about 200 nm. In some embodiments, the average diameter of the nanoparticles is about 180 nm. In any of the aforementioned embodiments, there may be little to no nanoparticle agglomeration.

In some embodiments, the average amount of the peptide comprising an amino acid sequence according to SEQ ID NO: 1 or SEQ ID NO: 2 is at least about 500 ng per mg of the nanoparticle composition. For example, the average amount of the peptide comprising an amino acid sequence according to SEQ ID NO: 1 or SEQ ID NO: 2 is at least about 500, 600, 700, 800, 900, 1000, 1100, 1200, 1300, 1400, 1500, 1600, 1700, 1800, 1900, or 2000 ng per mg of the nanoparticle composition, including all integers and ranges therebetween.

In some embodiments, the nanoparticles have a surface charge characterized by a zeta potential of between about 0 mV to about −30 mV. For example, the nanoparticles may have a surface charge characterized by a zeta potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, or about −30 mV. In some embodiments, the nanoparticles may have a surface charge characterized by a zeta potential ranging from about −0 mV to about −25 mV, about 0 mV to about −20 mV, about 0 mV to about −15 mV, about −0 mV to about −10 mV, about 0 mV to about −5 mV, about −5 mV to about −30 mV, about −5 mV to about −25 mV, about −5 mV to about −20 mV, about −5 mV to about −15 mV, about −5 mV to about −10 mV, about −5 mV to about −8 mV, about −10 mV to about −30 mV, about −10 mV to about −25 mV, about −10 mV to about −20 mV, about −10 mV to about −15 mV, about −10 mV to about −12 mV, about −15 mV to about −30 mV, about −15 mV to about −25 mV, about −15 mV to about −20 mV, about −15 mV to about −18 mV, about −20 mV to about −30 mV, about −20 mV to about −25 mV, about −20 mV to about −23 mV, about −25 mV to about −30 mV, or about −25 mV to about −28 mV. In some embodiments, the nanoparticles have a polydispersity index (PDI) of from about 0.120 to about 0.350. For example, the nanoparticles have a PDI of from about 0.120, about 0.130, about 0.140, about 0.150, about 0.160, about 0.170, about 0.180, about 0.190, about 0.200, about 0.210, about 0.220, about 0.230, about 0.240, about 0.250, about 0.260, about 0.270, about 0.280, about 0.290, about 0.300, about 0.310, about 0.320, about 0.330, about 0.340, to about 0.350 including all integers and ranges therebetween

In some embodiments, the nanoparticles may include additional additives (e.g. into the outer phase to increase encapsulation efficiency and create a dense, homogeneous sphere). For example, in some embodiments, the nanoparticles further comprise Zn²⁺ (e.g. ZnO) and/or Ca²⁺ (e.g. CaO), and/or Fe³⁺ (e.g. FeCl₃, Fe₂(SO₄)₃, Fe(NO₃)₃).

In some embodiments, the nanoparticles have a peptide load greater than about 10 ng peptide/μg particles. In some embodiments, the nanoparticles have a peptide load greater than about 20 ng peptide/μg particles, about 30 ng peptide/μg particles, about 40 ng peptide/μg particles, about 50 ng peptide/μg particles, about 60 ng peptide/μg particles, about 70 ng peptide/μg particles, about 80 ng peptide/μg particles, about 90 ng peptide/μg particles, about 100 ng peptide/μg particles, about 125 ng peptide/μg particles, about 150 ng peptide/μg particles, about 175 ng peptide/μg particles, about 200 ng peptide/μg particles, about 250 ng peptide/μg particles, about 300 ng peptide/μg particles, about 400 ng peptide/μg particles, or about 500 ng peptide/μg particles. Accordingly, in some embodiments, the nanoparticles have a loading capacity of about 1% to about 50%, or about 1% to about 25%, or about 1% to about 10%; or a loading capacity of about 1%, about 2%, about 3%, about 4%, about 5%, about 6%, about 7%, about 8%, about 9%, about 10%, about 15%, about 20%, about 25%, about 30%, about 35%, about 40%, about 45%, or about 50%.

In some embodiments, the nanoparticles exhibit a controlled peptide release profile such that the peptide is released over about 1 week, about 2 weeks, about 3, weeks, about 4 weeks, or longer. In some embodiments, the peptide release profile is such that about 50%, about 40%, about 30%, about 20%, about 15%, about 10%, about 9%, about 8%, about 7%, about 6%, about 5%, about 4%, about 3%, about 2%, or about 1% of the encapsulated peptide is released in the first 24 hours after administration. In some embodiments, less than about 50%, less than about 40%, less than about 30%, less than about 20%, less than about 15%, or less than about 10% of the encapsulated peptide is released in the first 24 hours. In further embodiments, the remaining encapsulated peptide or substantially all of the remaining encapsulated peptide is released over the following 2 days, 3 days, 4 days, 5 days, 6 days, 1 week, 2 weeks, 3 weeks, or 4 weeks. In some embodiments, the peptide release profile is substantially corresponding to the following pattern: at 24 hours, from about 5% to about 60% of the total encapsulated peptide is released; and at about 7, 14, 21, 28, or more days, from about 50% to about 100% of the total encapsulated peptide is released. Thus for example, at the 24 hour time point, less than about 60% of the total encapsulated peptide is released, and at the 21 day time point up to about 100% of the total encapsulated peptide is released. Accordingly, in some embodiments, at the 24 hour time point, less than about 60%, about 55%, about 50%, about 45%, about 40%, about 35%, about 30%, about 25%, about 20%, about 15%, about 10%, or about 5% of the total encapsulated peptide is released. In some embodiments, at the 14 day time point, up to about 50%, about 55%, about 60%, about 65%, about 70%, about 75%, about 80%, about 85%, about 90%. about 95%, or about 100% of the total encapsulated peptide is released. In some embodiments, at the 21 day time point, up to about 50%, about 55%, about 60%, about 65%, about 70%, about 75%, about 80%, about 85%, about 90%. about 95%, or about 100% of the total encapsulated peptide is released. In some embodiments, at the 28 day time point, up to about 50%, about 55%, about 60%, about 65%, about 70%, about 75%, about 80%, about 85%, about 90%. about 95%, or about 100% of the total encapsulated peptide is released. In some embodiments, there is an initial “burst” release of peptide in the first 24 hours, of less than about 50%, or about 10%, or less than about 10%; and a release of peptide over the following 1, 2, or 3 weeks of about 50%, 60%, 70%, 80%, 90%, or more peptide is released. It will be appreciated by the skilled artisan that the release characteristics can be tailored to a specific indication. For example, one target indication may be optimally treated with nanoparticles that fully release (e.g. about 80%-100%) the total encapsulated peptide by day 14, while a second target indication may be optimally treated with nanoparticles that fully release (e.g. about 80%-100%) the total encapsulated peptide by day 28. Thus, in some embodiments, the nanoparticles fully release (e.g. about 80%-100%) the total encapsulated peptide by day 7, day 8, day 9, day 10, day 11, day 12, day 13, day 14, day 15, day 16, day 17, day 18, day 19, day 20, day 21, day 22, day 23, day 24, day 25, day 26, day 27, day 28, day 29, day 30, or day 31.

In some embodiments, the nanoparticles may include different or additional stabilizers, surfactants, and/or emulsifiers. Suitable agents include, but are not limited to, polysorbates, alkyl sulfosuccinates, alkyl phenols, ethoxylated alkyl phenols, alkyl benzene sulfonates, fatty acids, ethoxylated fatty acids, propoxylated fatty acids, fatty acid salts, tall oils, castor oils, triglycerides, ethoxylated triglycerides, alkyl glucosides, and mixtures and derivatized fatty acids such as those disclosed in U.S. Pat. No. 6,849,581, incorporated by reference herein in its entirety. Suitable polysorbates include, but are not necessarily limited to, sorbitan monolaurate, sorbitan monopalmitate, sorbitan monostearate, sorbitan monooleate, sorbitan monodecanoate, sorbitan monooctadecanoate, sorbitan trioleate and the like and ethoxylated derivatives thereof. For instance, these agents may have up to 20 ethoxy groups thereon. Suitable polysorbates include, but are not necessarily limited to, SPAN® 40, SPAN 40, SPAN 60 and SPAN 80 polysorbates available from Croda International PLC. Other suitable agents include stearyl alcohol, lecithin, fatty acid amines, ethoxylated fatty acid amines and mixtures thereof. In one non-limiting embodiment, more than one agent is used.

In some embodiments, a composition suitable for freezing and/or storage is contemplated, including nanoparticles disclosed herein and a solution suitable for freezing, e.g. a sucrose and/or cyclodextrin solution is added to the nanoparticle composition. For example, the cryoprotectant may act to prevent the particles from aggregating upon freezing. A variety of suitable cryoprotectants will be readily apparent to a skilled artisan, and include, but are not limited to sucrose, trehalose, dextrose, or sorbitol.

### Pharmaceutical Formulations

In some embodiments, the present disclosure provides a pharmaceutical formulation comprising the nanoparticle composition of the present disclosure.

Pharmaceutical formulations comprising the nanoparticle composition of the present disclosure as described herein, may be formulated for delivery by any route that provides an effective dose of the nanoparticles or the amino acid sequence according to SEQ ID NO: 1 or SEQ ID NO: 2. Accordingly, the pharmaceutical compositions may be formulated for any appropriate manner of administration, including, for example, topical, oral, enteral, nasal (i.e., intranasal), inhalation, intrathecal, rectal, vaginal, intraocular, subconjunctival, buccal, sublingual, intrapulmonary, intradermal, intranodal, intratumoral, transdermal, or parenteral administration, including subcutaneous, percutaneous, intravenous, intramuscular, intrasternal, intracavernous, intrameatal, intratumoral, intracranial, intraspinal or intraurethral injection or infusion. The term parenteral as used herein includes iontophoretic (e.g., U.S. Pat. Nos. 7,033,598; 7,018,345; 6,970,739), sonophoretic (e.g., U.S. Pat. Nos. 4,780,212; 4,767,402; 4,948,587; 5,618,275; 5,656,016; 5,722,397; 6,322,532; 6,018,678), thermal (e.g., U.S. Pat. Nos. 5,885,211; 6,685,699), passive transdermal (e.g., U.S. Pat. Nos. 3,598,122; 3,598,123; 4,286,592; 4,314,557; 4,379,454; 4,568,343; 5,464,387; UK Pat. Spec. No. 2232892; U.S. Pat. Nos. 6,871,477; 6,974,588; 6,676,961), microneedle (e.g., U.S. Pat. Nos. 6,908,453; 5,457,041; 5,591,139; 6,033,928) administration and also subcutaneous injections, intravenous, intramuscular, intrastemal, intracavernous, intrathecal, intranodal, intrameatal, intraurethral, intratumoral injection or infusion techniques. Methods of administration are described in greater detail herein.

In some embodiments, the present disclosure provides a pharmaceutical formulation comprising the nanoparticle composition of the present disclosure and one or more pharmaceutically acceptable carriers or excipients.

Any physiologically or pharmaceutically suitable excipient or carrier (i.e., a nontoxic material that does not interfere with the activity of the active ingredient) known to those of ordinary skill in the art for use in pharmaceutical compositions may be employed in the compositions described herein. Exemplary excipients include diluents and carriers that maintain stability and integrity of proteins. Excipients for therapeutic use are well known, and are described, for example, in Remington: The Science and Practice of Pharmacy (Gennaro, 21st Ed. Mack Pub. Co., Easton, Pa. (2005)), and are described in greater detail herein.

“Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate buffered saline at physiological pH may be used. Preservatives, stabilizers, dyes and even flavoring agents may be provided in the pharmaceutical composition. For example, sodium benzoate, sorbic acid and esters of p hydroxybenzoic acid may be added as preservatives. Id. at 1449. In addition, antioxidants and suspending agents may be used. Id.

In some embodiments, the one or more pharmaceutically acceptable carriers or excipients are selected from the groups consisting of anti-adherents, antioxidants, binders, coatings, compression aids, disintegrants, dyes (colors), emollients, emulsifiers, fillers (diluents), film formers or coatings, flavors, glidants (flow enhancers), lubricants, preservatives, sorbents, suspending or dispersing agents, sweeteners, and waters of hydration. In some embodiments, the one or more pharmaceutically acceptable carriers or excipients are selected from the groups consisting of butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, and xylitol.

In some embodiments, the formulation may be a liquid formulated for injection. A liquid pharmaceutical formulation may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In particular embodiments, the use of physiological saline is used, and an injectable pharmaceutical composition is optionally sterile. In some embodiments, further comprising diluents such as buffers, antioxidants such as ascorbic acid, carbohydrates such as glucose, sucrose or dextrins, chelating agents such as EDTA, and glutathione.

In some embodiments, the formulation is formulated for oral administration. In some embodiments, an excipient and/or binder may be present. Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach

In some embodiments, the formulation is in the form of an aerosol, cream, foam, emulsion, gel, liquid, lotion, patch, powder, solid, spray, or any combinations thereof.

In some embodiments, the present disclosure provides a topical formulation comprising the nanoparticle composition of the present disclosure. In some embodiments, the topical formulation further comprises hydroxyethylcellulose gel. In some embodiments, the hydroxyethylcellulose gel stabilizes the alpha connexin peptide provided herein.

Pharmaceutical formulations comprising the nanoparticle composition of the present disclosure may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

In some embodiments, the provided pharmaceutically acceptable carrier is a poloxamer. Poloxamers, referred to by the trade name Pluronics®, are nonionic surfactants that form clear thermoreversible gels in water. Poloxamers are polyethylene oxide-polypropylene oxide-polyethylene oxide (PEO-PPO-PEO) tri-block copolymers. The two polyethylene oxide chains are hydrophilic but the polypropylene chain is hydrophobic. These hydrophobic and hydrophilic characteristics take charge when placed in aqueous solutions. The PEO-PPO-PEO chains take the form of small strands where the hydrophobic centers would come together to form micelles. The micelle, sequentially, tend to have gelling characteristics because they come together in groups to form solids (gels) where water is just slightly present near the hydrophilic ends. When it is chilled, it becomes liquid, but it hardens when warmed. This characteristic makes it useful in pharmaceutical compounding because it can be drawn into a syringe for accurate dose measurement when it is cold. When it warms to body temperature (when applied to skin) it thickens to a perfect consistency (especially when combined with soy lecithin/isopropyl palmitate) to facilitate proper injunction and adhesion. Pluronic® F127 (F127) is widely used because it is obtained easily and thus it is used in such pharmaceutical applications. F127 has a EO:PO:EO ratio of 100:65:100, which by weight has a PEO:PPO ratio of 2:1. Pluronic gel is an aqueous solution and typically contains 20-30% F-127. Thus, the provided compositions can be administered in F127.

In some embodiments, the topical formulation further comprises a buffering agent. Suitable buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate. Non-limiting examples of suitable buffering agents include aluminum, magnesium hydroxide, aluminum hydroxide/magnesium hydroxide co-precipitate, aluminum hydroxide/sodium bicarbonate co-precipitate, calcium acetate, calcium bicarbonate, calcium borate, calcium carbonate, calcium bicarbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). In addition, due to the ability of proteins or protein hydrolysates to react with stomach acids, they too can serve as buffering agents in the present embodiments. Furthermore, combinations or mixtures of the above mentioned buffering agents can be used in the pharmaceutical formulations described herein. In some embodiments, the buffering agent is a phosphate buffer. In some embodiments, the buffering agent maintains the pH of the topical formulation between about 5 and about 7. For example, the, the buffering agent maintains the pH of the topical formulation between about 5.0, about 5.1, about 5.2, about 5.3, about 5.4, about 5.6, about 5.7, about 5.8, about 5.9, about 6.0, about 6.1, about 6.2, about 6.3, about 6.4, about 6.5, about 6.6, about 6.7, about 6.7, about 6.8, about 6.9, or about Methods of Making

In some embodiments, the present disclosure provides a method of making the nanoparticle composition of the present disclosure, comprising the steps of

(a) combining a first solution comprising one or more biodegradable or biocompatible polymers dissolved in an organic solvent with a second solution comprising an amino acid sequence according to SEQ ID NO: 1 or SEQ ID NO: 2 dissolved in a first aqueous solvent;

(b) emulsifying the mixture of step (a);

(c) adding the emulsion of step (b) to a second solution comprising one or more biodegradable or biocompatible polymers dissolved in a second aqueous solvent;

(d) removing the organic solvent; and

(e) optionally purifying the product of (d).

In some embodiments, the method further comprises the step of (f) freezing and/or lyophilizing the product of (d) or (e). In some embodiments, step (d) is performed by stirring the mixture of (c) for an amount of time sufficient to remove the organic solvent from the mixture. In some embodiments, when step (d) is performed by stirring the mixture of (c) for an amount of time sufficient to remove the organic solvent from the mixture, the time is takes to remove the solvent from the mixture (e.g. via evaporation) may be about 15 minutes, about 30 minutes, about 45 minutes, about 1 hour, about one hour and 15 minutes, about one hour and 30 minutes, about one hour and 45 minutes, about two hours, about two hours and 15 minutes, about two hours and 30 minutes, about two hours and 45 minutes, about three hours, about four hours, about five hours, about six hours, about 9 hours, about 12 hours, about 15 hours, about 18 hours, about 21 hours, or about one day. In some embodiments, the amount of time sufficient to remove the organic solvent from the mixture is from about 1 minute to about 10 hours. In some embodiments, the amount of time sufficient to remove the organic solvent from the mixture is about 3 hours. The amount of time sufficient to remove the organic solvent from the mixture may vary based a number of factors, such as solvent removal technique, temperature and pressure, and the organic solvent. For example, in some embodiments, when step (d) is performed by stirring the mixture of (c) for an amount of time sufficient to remove the organic solvent from the mixture and the organic solvent is ethyl acetate is the solvent, the time is takes to remove the ethyl acetate from the mixture (e.g. via evaporation) may be about 15 minutes, about 30 minutes, about 45 minutes, about 1 hour, about one hour and 15 minutes, about one hour and 30 minutes, about one hour and 45 minutes, about two hours, about two hours and 15 minutes, about two hours and 30 minutes, about two hours and 45 minutes, about three hours, about four hours, about five hours, about six hours, about 9 hours, about 12 hours, about 15 hours, about 18 hours, about 21 hours, or about one day. In some embodiments, the amount of time sufficient to remove ethyl acetate from the mixture is from about 1 minute to about 10 hours. In some embodiments, the amount of time sufficient to remove ethyl acetate from the mixture is about 3 hours. In some embodiments, about 10, about 20, about 30, about 40, about 50, about 60, about 70, about 80, about 90, or about 100% of the organic solvent is removed in step (d). In some embodiments, step (d) is performed by rotary evaporation.

In some embodiments, step (a) is performed while being sonicated. The sonication may continue after the first solution and second solution are mixed. For example, the first solution and second solution may be mixed while the mixture is sonicated and the mixture may continue to be sonicated for a period of time afterwards. Thus, the total sonication time may range from a matter of seconds to greater than one hour. For example, the sonication time may be about 5 seconds, about 10 seconds, about 15 seconds, about 20 seconds, about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, to about 1 hour. In some embodiments, the total sonication time is about 2 minutes. In some embodiments, the sonication is probe sonication at 40% amplitude.

In some embodiments, step (c) is performed while being vortexted. As defined herein, “while being vortexed” means that the vortexing occurs intermittently while the emulsion is being added (e.g. in a drop-wise manner). For example, some of the emulsion of step (b) is added to the second solution, whereupon a vortexing step is performed and this process is repeated until all of the emulsion of step (b) is added. In some embodiments, step (c) comprises adding the emulsion of step (b) to a second solution comprising one or more biodegradable or biocompatible polymers dissolved in a second aqueous solvent followed by vortexing the mixture. The sonication may continue after the emulsion of step (b) and the second solution comprising one or more biodegradable or biocompatible polymers dissolved in a second aqueous solvent are added together. For example, the emulsion of step (b) and second solution comprising one or more biodegradable or biocompatible polymers dissolved in a second aqueous solvent may be added while the mixture is vortexed and the mixture may continue to be vortexed for a period of time afterwards. Thus, the total vortex time may range from a matter of seconds to greater than one hour. For example, the vortex time may be about 5 seconds, about 10 seconds, about 15 seconds, about 20 seconds, about 30 seconds, about 45 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, to about 1 hour. In some embodiments, the product of step (c) is sonicated prior to step (d). In some embodiments, the product of step (c) is sonicated during step (d). In some embodiments, the product of step (c) is sonicated prior to and during step (d).

A skilled artisan will recognize a variety of methods to purify the products of the present disclosure. For example, in some embodiments, the purifying of step (e) is performed by washing the product of (d). In some embodiments, the purifying of step (e) is performed by dialysis, evaporation, or sequential centrifugation.

In some embodiments, the product of any one of steps (c)-(f) are sterilized. Suitable sterilization methods will be readily apparent to a skilled artisan. For example, suitable sterilization techniques include, but are not limited to, heat sterilization, chemical sterilization, radiation sterilization, and filtration.

In some embodiments, the biodegradable or biocompatible polymers of step (a) is PLGA. In some embodiments, the biodegradable or biocompatible polymers of step (c) further comprises PVA.

A variety of organic solvents may be used in the methods of the present disclosure and are readily apparent to a skilled artisan. Suitable organic solvents include, but are not limited to, methanol, ethanol, ethyl acetate, isopropanol, methoxy propanol, butanol, DMSO, dioxane, DMF, NMP, THF, acetone, dichloromethane, toluene, or a mixture of two or more of the solvents. In some embodiments, the organic solvent is ethyl acetate.

The term “aqueous solvent” refers to a composition having water as the major component and that is a liquid at room temperature. In some embodiments, the first and second aqueous solvent is water.

In some embodiments, the present disclosure provides a method of making the nanoparticle composition of the present disclosure, comprising

(a) providing
i. an amino acid sequence according to SEQ ID NO: 1 or SEQ ID NO: 2 and one or more biodegradable or biocompatible polymers dissolved in an water-miscible organic solvent; and
ii. an anti-solvent;

(b) mixing amino acid sequence according to SEQ ID NO: 1 or SEQ ID NO: 2 and one or more biodegradable or biocompatible polymers dissolved in the water-miscible organic solvent with the anti-solvent, such that the nanoparticle composition is formed; and

(c) optionally purifying the product of (b).

In some embodiments, the method further comprises the step of (d) freezing and/or lyophilizing the product of (b) or (c).

In some embodiments, the mixing is performed with a jet mixer. For example, the jet mixer may be a confined impinging jets (CIJ) mixer containing 2 jets or a multi-inlet vortex mixer (MIVM) containing up to 4 jets. Thus, in some embodiments, the jet mixer is a 2-jet mixer. In some embodiments, the jet mixer is a 4-jet mixer. In some embodiments, the mixing is performed with a coaxial turbulent jet mixer, a Roughton mixer, a tee mixer, a vortex mixer or a miniature, micro, or handheld CIJ and MIVM mixer.

A variety of water-miscible organic solvents may be used in the context of the present disclosure. The term “water-miscible organic solvent” refers to a solvent other than water that readily forms a homogenous solution with water at room temperature and at atmospheric pressure. Examples of suitable water-miscible organic solvents include ethanol, methanol, isopropanol, acetonitrile, dimethylformamide, dimethyl sulfoxide (DMSO) and formic acid. In some embodiments, the water-miscible organic solvent is DMSO.

As used herein, the term “anti-solvent” refers to a solvent in which the solvent used to dissolve the polymer is also miscible with the anti-solvent, but the polymer does not dissolve in the anti-solvent. In some embodiments, water is used as an “anti-solvent”. Without being bound by any particular theory, when the two solvents are mixed, the polymer precipitates out of solution, capturing the amino acid sequence according to SEQ ID NO: 1 or SEQ ID NO: 2 in the process.

In some embodiments, the biodegradable or biocompatible polymers of step (a) is PLGA. In some embodiments, the biodegradable or biocompatible polymers of step (a) further comprises PVA.

In some embodiments, the nanoparticle encapsulation efficiency of the amino acid sequence is greater than about 20%. For example, the nanoparticle encapsulation efficiency of the amino acid sequence is greater than about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

The methods surprisingly resulted in nanoparticles with a uniform particle size and little to no agglomeration. Uniform particle size and little to no agglomeration is desirable because it eliminates the need for costly processing steps such as wet and/or dry milling steps. For example, in some embodiments, the average diameter of the nanoparticles is between about 100 nm and about 300 nm. In some embodiments, the average diameter of the nanoparticles is between about 100 nm and about 200 nm. In some embodiments, the average diameter of the nanoparticles is about 180 nm. In any of the aforementioned embodiments, there may be little to no nanoparticle agglomeration. In some embodiments, the methods produce nanoparticles that do not require costly processing steps, such as wet or dry milling.

In some embodiments, the present disclosure provides a method of manufacturing a topical formulation comprising:

a) mixing propylene glycol, glycerin, methylparaben and propylparaben until the parabens are completely dissolved;

b) separately mixing purified water, EDTA, monobasic sodium phosphate, dibasic sodium phosphate and D-mannitol until a clear solution is obtained;

c) adding the solution from a) to the solution from b), rinsing the container of the solution from a) with purified water, adding the rinse to the combined solutions, and mixing until the combined solutions are visually homogeneous;

d) with homogenization mixing, adding hydroxyethyl cellulose into the combined solutions of c) and mixing until the polymer is fully dispersed;

e) separately mixing purified water with an amino acid sequence according to SEQ ID NO: 1 or SEQ ID NO: 2 until the peptide is completely dissolved;

f) adding the solution from e) to the solution of d), rinsing the container of the solution from e) with purified water, adding the rinse to the combined solutions, and mixing until the combined solution are homogeneous.

### Methods of Treatment

In some embodiments, the present disclosure provides a method of treating cancer in a patient in need thereof wherein the method comprises, administering to the patient a therapeutically effective amount of the pharmaceutical formulations of the present disclosure.

A variety of cancers may be treated by the compositions of the present disclosure. For example, in some embodiments, the cancer is selected from the group consisting of glioma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma, glioblastoma, ovarian cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers, testicular cancer, colon and rectal cancers, prostatic cancer, pancreatic cancer, rhabdomyosarcoma, spinal cord tumors, and bone cancers such as osteosarcoma, Ewing's sarcoma, chondrosarcoma, multiple myeloma and giant-cell bone tumor. In some embodiments, the cancer is glioma. In some embodiments, the glioma is glioblastoma.

The pharmaceutical formulations of the present disclosure may be administered by any method that provides an effective dose of the nanoparticles or the amino acid sequence according to SEQ ID NO: 1 or SEQ ID NO: 2. Accordingly, the pharmaceutical formulations may be administered by any of the following routes: topical, oral, enteral, nasal (i.e., intranasal), inhalation, intrathecal, rectal, vaginal, intraocular, subconjunctival, buccal, sublingual, intrapulmonary, intradermal, intranodal, intratumoral, transdermal, or parenteral administration, including subcutaneous, percutaneous, intravenous, intramuscular, intrasternal, intracavernous, intrameatal, intratumoral, intracranial, intraspinal or intraurethral injection or infusion. In some embodiments, the pharmaceutical formulation is administered via an injection. For example, the pharmaceutical formulation is administered by subcutaneous, intradermal, intramuscular, intratumoral, or intravenous injection. In some embodiments, the pharmaceutical formulation is administered by intratumoral injection.

Recent studies indicate that the gap junction protein connexin 43 (Cx43) renders GBM cells resistant to TMZ through its carboxyl terminus (CT) (Murphy et al., Cancer Res. 76:139-49 (2016). In some embodiments, the alpha connexin polypeptide nanoparticle formulations provided herein counteract the resistance of TMZ or other chemotherapeutic agents by inhibiting alpha connexin activity.

Thus, in some embodiments, the method further comprises administering a chemotherapeutic agent (e.g. TMZ). A variety of chemotherapeutic agents may be used and will be readily apparent to the skilled artisan. Suitable chemotherapeutic agents include, but are not limited to: toxins (e.g., saporin, ricin, abrin, ethidium bromide, diptheria toxin, and Pseudomonas exotoxin); taxanes; alkylating agents (e.g., temozolomide (TMZ), nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes (e.g., cisplatin, carboplatin, tetraplatin, ormaplatin, thioplatin, satraplatin, nedaplatin, oxaliplatin, heptaplatin, iproplatin, transplatin, and lobaplatin); bioreductive alkylators such as mitomycin, procarbazine, dacarbazine and altretamine); DNA strand-breakage agents (e.g., bleomycin); topoisomerase II inhibitors (e.g., amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, and teniposide); DNA minor groove binding agents (e.g., plicamydin); antimetabolites (e.g., folate antagonists such as methotrexate and trimetrexate); pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); anthracyclines; and tubulin interactive agents (e.g., vincristine, vinblastine, and paclitaxel (Taxol®)), and combinations thereof. In some embodiments, the chemotherapeutic agent is TMZ.

In some embodiments, the alpha connexin peptide-nanoparticle compositions provided herein sensitize tumors to treatment with another therapy, such as a chemotherapeutic agent. In some embodiments, the alpha connexin peptide-nanoparticle compositions provided herein increase the effectiveness of another cancer therapy, such as a chemotherapeutic agent, radiation therapy, or surgery. Thus, in some embodiments, the present disclosure provides compositions and methods for treating cancer by administering the alpha connexin peptide-nanoparticle compositions provided herein in combination with another cancer therapy in order to enhance the effectiveness of the other cancer therapy. For example, in some embodiments, the alpha connexin peptide-nanoparticle compositions provided herein sensitize tumors to TMZ treatment. In some embodiments, the alpha connexin peptide-nanoparticle compositions provided herein restore the sensitivity of tumors to TMZ treatment. In some embodiments, the alpha connexin peptide-nanoparticle compositions provided herein maintain the sensitivity of tumors to TMZ treatment.

The pharmaceutical formulations may be administered over multiple doses over the course of treatment. For example, in some embodiments, the pharmaceutical formulations are administered (e.g. via intratumoral injection) 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 times over the course of treatment. Accordingly, the course of treatment may last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the pharmaceutical formulations are administered (e.g. via intratumoral injection) on consecutive days. In some embodiments, the pharmaceutical formulations are administered (e.g. via intratumoral injection) on alternating days (e.g. every other day).

In some embodiments, the chemotherapeutic agent (e.g. TMZ) is administered concomitantly with the pharmaceutical formulations of the present disclosure. For example, in some embodiments, the chemotherapeutic agent is administered on the same day(s) as the pharmaceutical formulation.

In some embodiments, the chemotherapeutic agent (e.g. TMZ) is not administered concomitantly with the pharmaceutical formulations of the present disclosure. For example, in some embodiments, the chemotherapeutic agent is administered on a different day(s) than the pharmaceutical formulation.

In some embodiments, the present disclosure provides a method of treating a chronic wound in a subject, comprising administering to the subject the topical formulation of the present disclosure, wherein the formulation is administered in a dosing regimen effective for the treatment of the chronic wound. In some embodiments, the formulation is administered daily or weekly. In some embodiments, the formulation is administered in a dosing regimen at day 0, day 3, week 1, week 2, week 3, week 4, week 5, week 6, week 7, week 8, week 9, week 10, week 11, and week 12, wherein the symptoms of the chronic wound are reduced. In a further embodiment, the formulation does not induce excessive levels of side effects. In another embodiment, the chronic wound is improved in the absence of clinically significant abnormalities. In another embodiment, the method reduces the time to 100% wound closure, as compared to the time to 100% wound closure when the standard of care treatment is used. In other embodiments, the method reduces the time to 100% wound closure when administered in conjunction with standard of care, as compared to treatment with either standard of care alone. In one embodiment, the method reduces the time to 50% wound closure, as compared to the time to 50% wound closure when the standard of care treatment is used. In another embodiment, the percent wound closure at 4 weeks is higher in subjects treated with the methods and formulations disclosed herein, as compared to the percent wound closure at 4 weeks in subjects treated with standard of care treatment.

In another embodiment, the method results in a reduction in pain levels in the subject. In a further embodiment, the pain level is determined through patient self-assessment. In one embodiment, the method increases the average percent of wound closure at 12 weeks, 11 weeks, 10 weeks, 9 weeks, 8 weeks, 7 weeks, 6 weeks, 5 weeks, 4 weeks, 3 weeks, or 2 weeks as compared to standard of care treatment. In one embodiment, the method increases the average percent of wound closure at 12 weeks. In one embodiment, the method decreases the wound area as compared to the wound area in subjects that are treated with standard of care therapy. In one embodiment, the method does not induce the production of anti-alpha connexin polypeptide antibodies in the subject.

In another embodiment, the method increases the incidence or frequency of 100% complete wound closure compared to standard of care treatments for wound healing. In another embodiment, the method is used to treat an ulcer lacking sufficient wound size reduction within one, four, 12, 24, 36, or more weeks of standard of care. In one embodiment, the method is used to treat an ulcer with less than 50% wound closure within one four, 12, 24, 36, or more weeks of standard of care. In one embodiment, the method is used to treat an ulcer lacking sufficient wound size reduction within one, four, 12, 24, 36, or more weeks of standard of care. In one embodiment, the method is used to treat an ulcer lacking sufficient wound size reduction within one, four, 12, 24, 36, or more weeks of standard of care. In one embodiment, the method is used to treat a patient with an ulcer possessing wound area and duration characteristics of a chronic wound. In one embodiment, the method is used to treat a patient with an ulcer possessing a wound with a non-healing wound trajectory. In one embodiment, the method is used to treat an ulcer possessing wound area and time duration characteristics of a chronic wound.

In some embodiments, the present disclosure provides a method of treating a chronic wound in a subject, comprising administering to the subject a topical formulation of the present disclosure in addition to standard of care compression therapy, wherein the chronic wound is healed at a faster rate and/or increased frequency than achieved with standard of care compression therapy alone.

In some embodiments, the formulation for use in treatment of a chronic wound comprises at least one alpha connexin polypeptide and hydroxyethylcellulose gel. In a further embodiment, the hydroxycellulose gel is present at a concentraiton of about 2%, about 1.75%, about 1.5%, about 1.25%, about 1.0%, or about 0.75%. In one embodiment, the hydroxycellulose gel is present at a concentration of about 1.25% (w/w).

In some embodiments, the chronic wound is an ulcer. In a further embodiment, the chronic wound is a lower extremity ulcer. In another embodiment, the chronic wound is selected from the group consisting of venous leg ulcers, diabetic foot ulcers, and pressure ulcers.

In some embodiments, the chronic wound is an ulcer (e.g. a lower extremity ulcer). In some embodiments, the chronic wound is selected from the group consisting of venous leg ulcers, diabetic foot ulcers, and pressure ulcers.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

### EXAMPLES

The following examples are provided to illustrate the present disclosure, and should not be construed as limiting thereof. In these examples, all parts and percentages are by weight, unless otherwise noted. Abbreviations in the examples are noted below.

### Example 1: Poly(Lactic-Co-Glycolic Acid) Nanoparticles for Controlled Delivery of a Peptide Via Double Emulsion-Solvent Evaporation Method

PLGA is used in small molecule drug delivery for a variety of applications.²⁻⁴ PLGA is useful for controlled-release of small molecules because it degrades over several weeks by hydrolysis through cleavage of its backbone ester linkages, forming biologically compatible byproducts, lactic acid and glycolic acid, which are readily metabolized by the body through the Krebs cycle and eliminated as dioxide carbon and water.^{2, 4}

Many studies have been successful in encapsulating hydrophilic and hydrophobic drugs using PLGA, especially small molecules, but peptides and proteins are significantly more challenging to encapsulate, especially while keeping a minimal particle size.¹³⁻¹⁸ While there are several methods to create nanoparticles, one of which is the emulsion-solvent evaporation method.¹⁹ The emulsion-solvent evaporation method involves first dissolving the polymer in a volatile, water-immiscible solvent before emulsifying in water with a surfactant, then allowing the solvent to evaporate. Hydrophobic drugs are generally encapsulated via a single emulsion process as described above (o/w), while hydrophilic drugs use a double emulsion (w/o/w) method.^{2, 19}

Effective therapeutic delivery of peptide and protein drugs is also challenged by short in vivo half-lives due to rapid degradation. Sustained release formulations of αCT1, a 25 amino acid peptide drug, was proposed to afford lower dosing frequency in indications that require long term treatment, such as chronic wounds and cancers. In this study, rhodamine B was used as a model drug to develop and optimize a double emulsion-solvent evaporation method of PLGA nanoparticle synthesis. Encapsulation of αCT1 in these nanoparticles resulted in a sustained in vitro release profile over three weeks, characterized by an initial burst release over the first three days followed by sustained release of up to 73% of total encapsulated drug over the remaining two and a half weeks.

To allow for a more comprehensive investigation, drug loading using a double emulsion process was compared with a single emulsion process.

### Materials and Methods

General. All purchased materials were used without further purification. PLGA (poly(D,L-lactide-coglycolide; 7000-17000 MW, 50:50 lactic acid:glycolic acid, acid terminated), PVA (poly(vinyl alcohol); 13000-23000 MW, 87-89% hydrolyzed), rhodamine B (RhB; HPLC grade, ≥95%), phosphate buffered saline powder (PBS; reconstituted in DI-water; BioPerformance Certified, pH 7.4), sucrose (BioUltra, for molecular biology, ≥99.5% (HPLC)), trehalose (Pharmaceutical Secondary Standard, certified reference material), and bovine serum albumin (BSA; essentially fatty acid free and essentially globulin free, ≥99%, agarose gel electrophoresis) were purchased from Sigma Aldrich. Ethyl acetate (EA; HPLC grade) was purchased from Fisher Scientific. The peptide drug, α-connexin carboxyl-terminal (αCT1) peptide was synthesized by the American Peptide Company (now Bachem; Sunnyvale, Calif.). The αCT1 peptide corresponds to a short sequence at the Connexin43 C-terminus RPRPDDLEI) linked to an antennapedia internalization sequence (RQPKIWFPNRRKPWKK).

Synthesis of Single Emulsion Particles. Single emulsion nanoparticle (SE-NP) synthesis method was modified from Mathew et al., 2012.¹² Briefly, after dissolving PLGA in EA, 0.1 mg of RhB was added directly to the PLGA solution. The solution was vortexed and then added to 1 mL of 2.5 w/v % solution of PVA in water. The solution was probe sonicated for 2 minutes at 40% amplitude. The solution was immediately added to 50 mL of 0.3 w/v % PVA in water, then was stirred for at least three hours to allow for EA evaporation.

SE-NPs were loaded with rhodamine B to test the difference in amount of drug loaded in the particles between single and double emulsion methods. RhB was used as a model drug for finding trends in drug loading because of its ease in concentration characterization. Furthermore, RhB and αCT1 share similar physiochemical characteristics including a positive overall charge. While the sizes of the molecules are different and could create differences in particle size, trends in loading and interactions with PLGA should be similar because of their charges. BSA is used to more closely mimic the size of the peptide. While much larger than αCT1 (MW 66.5 kDa compared with 3.5 kDa), it is on the extreme of bulkiness and should easily show trends in particle size during process modifications.

Synthesis of Double Emulsion Particles. The applied synthesis protocol was modified from Mathew et al. and Zhang et al.^{12, 18} Briefly, the double emulsion particles (DE-NPs) containing αCT1 (αCT1-NPs) were synthesized at room temperature by first dissolving 0.025 g of PLGA in 1 mL of EA for 30 minutes while vortexing intermittently. 50 μL of a 2 mmol αCT1 solution in water was then added and sonicated using a Qsonica Q55 probe sonicator at 40% amplitude for 2 minutes. The primary emulsion was then immediately added dropwise to 1 mL of 2.5 w/v % solution of PVA in water while vortexing. This mixture was then immediately probe sonicated again at 40% amplitude for 2 minutes. The double emulsion was then transferred to 50 mL of 0.3 w/v % PVA solution in water and stirred for at least three hours to allow for ethyl acetate evaporation. For rhodamine b or BSA nanoparticles (RhB-NPs, BSA-NPs), the same protocol was followed substituting a 2 mmol solution of rhodamine B or BSA in water for the αCT1 solution. Particles to be used in cell culture experiments were sterilized by filtration using Fisherbrand 25 mm nylon sterile syringe filters with a 0.2 μm pore size or made in a sterile biosafety cabinet during particle synthesis with all materials sterilized before use. Particles were then washed via the centrifugation method three times to remove excess PVA, frozen at ≤−20° C., and lyophilized, then stored at −20° C.

Materials Characterization Methods. Scanning electron microscopy (SEM) was performed at the Nanoscale Characterization and Fabrication Laboratory in Blacksburg, Va. using the LEO (Zeiss) field-emission SEM. Analysis of SEM images was completed using ImageJ software. Dynamic light scattering (DLS) and zeta potential were measured using a Malvern Zetasizer Nano-ZS. UV-vis absorbance for rhodamine release studies was measured using a Cary 60 UV-Vis spectrophotometer by Agilent Technologies.

In Vitro Release ofDrugfrom Nanoparticles. Lyophilized nanoparticles were resuspended in 0.01 M PBS solution at 1 mg/mL and incubated at 37° C. for the duration of the release study. At each time point, the particles were centrifuged into a pellet and the supernatant was collected for drug content analysis. The removed supernatant was replaced with the same volume of fresh PBS solution in order to maintain a consistent concentration and the particles were redispersed via bath sonication for 10-15 minutes. Samples of dispersions were taken and analyzed for dynamic light scattering (DLS). SEM samples were prepared by adding a drop of the particle suspension onto silicon wafer pieces taped to an SEM stub for future analysis.

Cell Culture. The human glioblastoma (GBM) cell line SF295 was maintained in Dulbecco's modified Eagle medium (Thermo Scientific) supplemented with 10% fetal bovine serum (Atlas Biologicals, Inc.), streptomycin (100 μg/ml) and penicillin (100 IU/ml). Human GBM stem cells (GSCs) VTC-037, isolated from a GBM patient who received surgery at the Carilion Clinic, as described previously,⁹ and LN229/GSC were maintained in Dulbecco's modified Eagle medium supplemented with Gibco® B-27® Supplements (Thermo Scientific), fibroblast growth factor (ProSpec-Tany TechnoGene Ltd., 20 ng/ml), and epidermal growth factor (ProSpec-Tany TechnoGene Ltd., 20 ng/ml).

Enzyme Linked Immunoassay (ELISA). To enable peptide tracking in in vitro and in vivo assays, an amino-terminal biotin tag was added to the αCT1 sequence. The in vitro release of biotin-tagged αCT1 was measured by sandwich enzyme linked immunoassay (ELISA) using the OptEIA kit (BD Biosciences). Each well of a Nunc MaxiSorp™ 96-well microplate (Thermo Scientific) was coated with coating buffer containing 1 μg/mL of anti-C-terminus connexin43 antibody (Sigma-Aldrich) and incubated overnight at 4° C. The wells were then washed before blocking with 1% bovine serum albumin for 2 h at room temperature. Serial dilution of biotin-tagged αCT1 as standards and samples containing biotin-tagged αCT1 collected at different times from the in vitro release study were added in the corresponding wells and incubated overnight at 4° C. The wells were then washed before adding 1 μg/ml Neutravidin-conjugated HRP (Thermo Scientific) for 1 h at room temperature. After washing, 3,3′,5,5′-Tetramethylbenzidine (TMB) chromogenic substrate solution was added and reacted for 10 min at room temperature in the dark, and the absorbance was measured at OD650 using a microplate reader (Molecular Devices). The reaction was then stopped by the addition of 2 M sulfuric acid and the absorbance was measured at OD450 using a microplate reader. All measurements were conducted in triplicate.

Cell Imaging and Immunofluorescence. Cells were seeded in 6-well plates or 35-mm glass-bottomed dishes (Mat-Tek) and RhB-NPs or αCT1-NPs filtered through 0.45 mM pores before lyophilization were resuspended in PBS and added to the medium at different concentrations. After overnight incubation, cells were then washed five times with PBS, replenished with fresh medium and observed at various times by phase-contrast and fluorescence microscopy using the EVOS™ FL Auto Imaging System (Thermo Scientific), or fixed at various times with 4% paraformaldehyde for 20 minutes and permeabilized with 0.1% Triton X-100 in a 3% BSA blocking solution for 2 hours at room temperature. Immunostaining was conducted with anti-C-terminus Connexin 43 antibody (Sigma-Aldrich, 1:3000) and detected using secondary antibody conjugated to Alexa Fluor® 488 (Thermo Scientific, 1:500). Biotin-tagged αCT1 was detected with Streptavidin conjugated to Alexa Fluor® 647 (Thermo Scientific, 1:500). Wheat Germ Agglutinin (WGA) conjugated to Alexa Fluor® 488 (Thermo Scientific, 1:500) was used to stain cell membranes. Slides were mounted using ProLong Gold anti-fade reagent with DAPI (Thermo Scientific). Cells were examined under an Opterra inverted fluorescence confocal microscope (Bruker).

### Results

Particle Size Optimization. PLGA nanoparticles encapsulating rhodamine B and αCT1 were synthesized. The initial double emulsion particles were made by using 0.05 g PLGA and 2 mL of EA in the primary emulsion and 5 w/v % PVA in the secondary emulsion. However, due to filtering requirements of 0.2 μm for sterilization purposes, the synthesis process required optimization in order to decrease particle size until the majority of the particles were below 0.2 μm. The amount of PLGA, ethyl acetate, and PVA were modified in order to optimize the size of the particles. An ice bath was also added in order to keep the PLGA below its glass transition temperature (T_{g}) during high-energy sonication to prevent NP coalescence. Finally, in order to better mimic how αCT1 may change the particle size during size optimization of the particles, 2 mM BSA in water was used as the drug mimic because of its bulkiness.

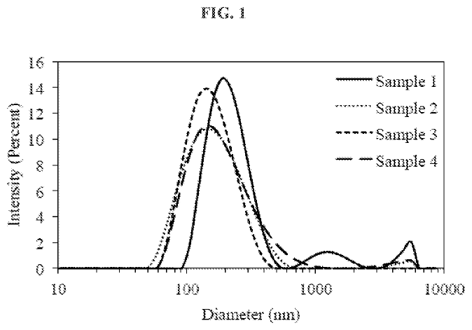

Table 7 gives a description of the parameters that were modified during the optimization. Because the peak diameter of single emulsion particles was generally below 200 nm, no optimization steps were conducted for the single emulsion particles. Dynamic light scattering (DLS; Malvern Zetasizer Nano ZS) was used to detect average nanoparticle diameter (FIG. 1). The original parameters used to make particles (Table 7, Sample 1) showed the largest average diameter at 229 nm. Small batch sizes and a lower concentration of PVA in the outer phase during emulsification yielded the smallest average diameter size with the lowest polydispersity index (PDI), meaning these smaller particles were more homogenous in size compared with other samples, and more than half of the collected particles can pass through a sterilization-sized filter (0.2 μm). Parameters from Sample 3 resulted in the smallest particles and PDI, so Sample 3 parameters were applied in the synthesis of double emulsion particles.

TABLE 7
Particle size optimization of double emulsion particles
using bovine serum albumin.

| | | | | w/v % | | Dia- | |
|---|---|---|---|---|---|---|---|
| | | | | PVA | | meter | |
| | | | | in | | (nm) | PDI |
| Sam- | BSA | PLGA | EA | outer | Ice | (avg ± | (avg ± |
| ple | (μL) | (g) | (mL) | phase | bath | stdev) | stdev) |
| 1 | 100 | 0.05 | 2 | 5 | No | 229 ± 4 | 0.301 ± 0.014 |
| 2 | 50 | 0.025 | 1 | 5 | Yes | 149 ± 1 | 0.190 ± 0.019 |
| 3 | 50 | 0.025 | 1 | 2.5 | Yes | 143 ± 1 | 0.158 ± 0.014 |
| 4 | 50 | 0.025 | 1 | 20 | Yes | 165 ± 1 | 0.219 ± 0.004 |

BSA = bovine serum albumin,
PLGA = poly(lactic-co-glycolic acid),
EA = ethyl acetate,
PVA = poly(vinyl alcohol),
Dia = diameter,
stdev = standard deviation,
PDI = polydispersity index.

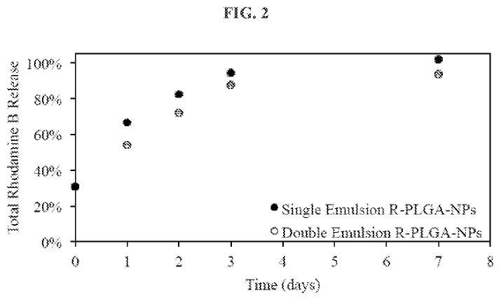

RhB Loading Content and Efficiency Comparison between Single and Double Emulsions. Next, an initial estimation of drug loading and release was made on both single emulsion and double emulsion particles. Table 8 compares the loading content and encapsulation efficiency of the single and double emulsion particles. The drug content, calculated using Eq. 1, was similar for both single and double emulsions. However, drug entrapment, calculated using Eq. 2, was higher for double emulsions compared with single emulsions. FIG. 2 also shows the release profiles of the drug from single and double emulsion particles. Over seven days, particles produced using single emulsion synthesis had a higher burst-release of rhodamine, releasing to completion more quickly than the double emulsion particles, which released 94% of rhodamine after seven days.

TABLE 8
Drug loading and encapsulation efficiency of RhB-PLGA-NPs (all particles filtered to 0.2 μm
before measuring loading).

| | RhB Loading (ng | RhB Loading | Encapsulation Efficiency |
|---|---|---|---|
| Sample | drug/mg particles) | (% ± st.dev) | (% ± st.dev) |
| RhB-PLGA-NPs, | 160 ± 53 | 0.0160 ± 0.0053 | 0.00028 ± 0.00015 |
| SE | | | |
| RhB-PLGA-NPs, | 167 ± 9 | 0.0167 ± 0.0009 | 0.0018 ± 0.0004 |
| DE | | | |

Double emulsion particles had a higher encapsulation efficiency and took longer to release all of the encapsulated material. As the goal is to develop a sustained release αCT1 formulation, these results supported further evaluation of the double emulsion particles.

$$\begin{matrix}Drug Loading ( w \text{/} w \% ) = \frac{Mass of Drug in NPs}{Mass of Recovered NPs} * 100 \% & Eq . 1 \\ Encapsulation Efficiency ( \% ) = \frac{Mass of Drug in NPs}{Mass of Drug used in Formulation} * 100 \% & Eq . 2\end{matrix}$$

Cryoprotectants. Lyophilization is used for long-term storage of the particles in order to keep the particles dry. RhB-NPs and αCT1-NPs were kept frozen to keep the particles below PLGA's T_{g} and to reduce possible exposure to moisture, which would prematurely degrade the particles. In order to lyophilize, particles in solution must first be frozen. Before use in clinical trials, the particles are to be resuspended in DI-water or PBS buffer solution and sterilized by filtration at 0.2 μm if particles were not synthesized in a sterile cabinet.

It was noticed that after lyophilization and resuspension, a significant portion of particles were lost during sterile filtration procedures. Even though the zeta potential of the rhodamine-loaded NPs after lyophilization (−28±2 mV) implied a stable suspension, the size of the particles increased during the freezing and/or drying process. It has been hypothesized that freezing allows ice crystals to puncture neighboring particles and essentially fuse them together to make a larger particle. Various methods have been explored to protect PLGA nanoparticles during freezing with positive results, but in general cryoprotectants such as small sugars (sucrose, trehalose, dextrose, sorbitol, etc.) are most commonly used.²⁰⁻²³ In this study, fast freezing via liquid nitrogen and the addition of cryoprotectants trehalose and sucrose were used to limit particle size growth.

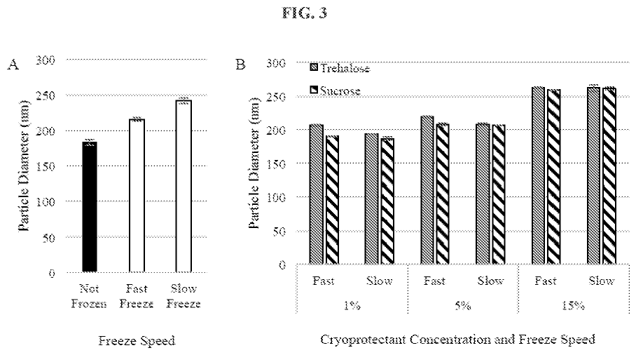

FIG. 3 shows a particle diameter of 183 nm before freezing. A slow freeze without cryoprotectant increased particle diameter to above 240 nm and a fast freeze limited growth to reach an average of 215 nm. When large amounts of cryoprotectant are added, such as at 15 w/v % in solution, particle size increased above the size of slow freezing without cryoprotectants, at around 260 nm. Adding large amounts (10% or greater) has in some cases been shown to increase particle size above that of particles frozen without cryoprotectant.^{21, 23} In this study, adding cryoprotectants in smaller amounts, especially at 1 w/v %, limited particle size growth. When trehalose was used at 1 w/v % and frozen quickly, particle size dropped below that of particles frozen with a fast freeze alone. When sucrose is added at 1 w/v %, with either fast or slow freezing, the average particle size remained the closest to the original particle size. The addition of trehalose at 1 w/v % and a slow freeze resulted in a similar particle size to methods using sucrose at the same concentration. Moving forward, 1 w/v % sucrose was added to all particle suspensions before freezing and lyophilizing.

Loading and Degradation of αCT1-NPs. After optimizing cryoprotectant parameters to reduce change in NP size during storage, loading and degradation studies were completed on αCT1-NPs. Particles were filtered to 0.2 μm before analysis. Drug loading by mass was 962±88 ng drug/mg particles, % loading was 0.0962±0.0088%, and encapsulation efficiency was 0.000966±0.00049%. Error is the standard deviation of at least three samples. Zeta potential of αCT1-NPs after lyophilization was −23 mV. Entrapment of αCT1 appears to be higher than that for RhB-NPs at almost 1 μg/mg particles, but percent drug loading is less than 0.1% for RhB-NPs and αCT1-NPs.

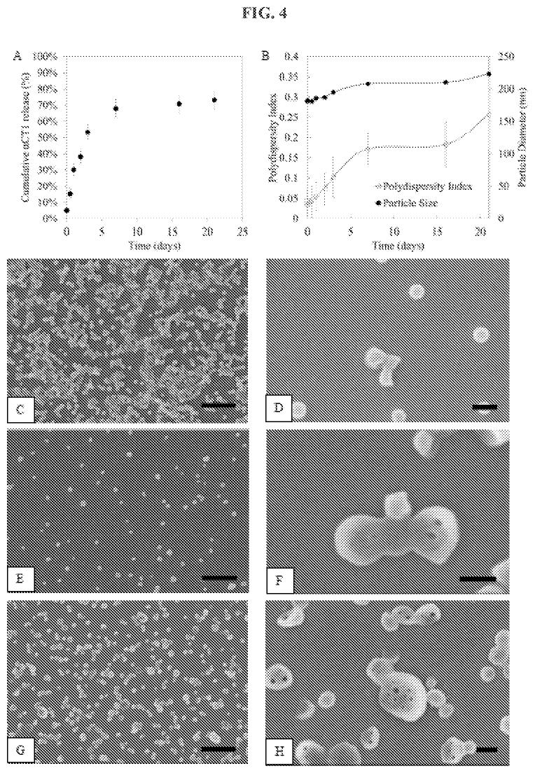

FIG. 4A shows the cumulative release of αCT1 over 21 days, where measurements were made via sandwich ELISA. The release profile showed a burst effect, in which about 50% of peptide was released after three days, followed by a sustained release over the subsequent 18 days until 73% of the total encapsulated drug was released.

Particle morphology and diameter changes were monitored over time using measurements from DLS and SEM image analysis via ImageJ software. Particle size diameters measured using DLS were approximately twice the size of measurements made via SEM. Both DLS and SEM showed increasing particle size from 186 to 208 over the course of 7 days, then increased in size again between 2 and 3 weeks to 223 nm. FIG. 4B shows the particle size and polydispersity index (PDI) of the particles over time during the degradation study as measured by DLS. PDI also increased with time. Similar particle size trends were found with measurements by SEM. FIG. 4C-H shows SEM images at Day 1, Day 7, and Day 21 of the degradation. Pores or holes seem to appear after Day 7, and increase in size and quantity with time.

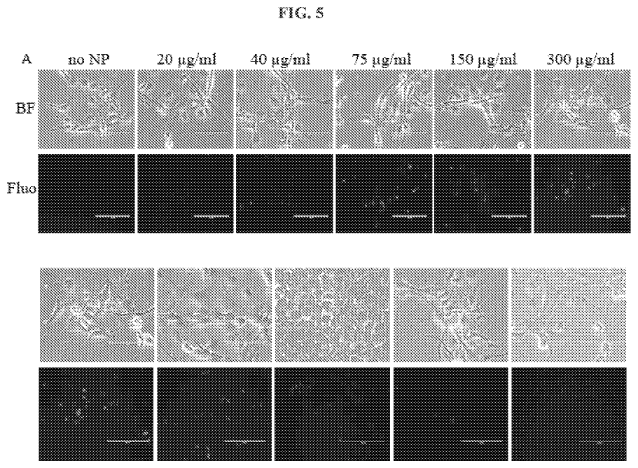

RhB-NP and αCT1-NP Uptake in GSCs and GBM cells. RhB-NPs added to VTC-037 GSCs at various concentrations showed that at least 300 μg/mL of NPs could be added to cells without affecting cell plate adhesion (FIG. 5A). This concentration was then used for a three-week cell culture to study degrading RhB-NPs and their effect on cells and how long RhB may remain in the cells over time (FIG. 5B). For the first week of release, a large amount of RhB is present in the cells. At 14 and 21 days, RhB signal is still detected in the cells even after cell passage through trypsinization at day 10.

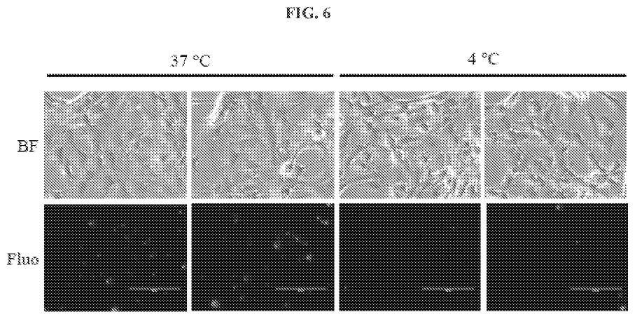

When 200 μg/ml of RhB-NPs were added to VTC-037 GSCs, cells incubated at 37° C. showed uptake of the RhB-NPs similar to FIG. 5, but cells incubated at 4° C. had reduced cellular uptake of the RhB-NPs (FIG. 6).

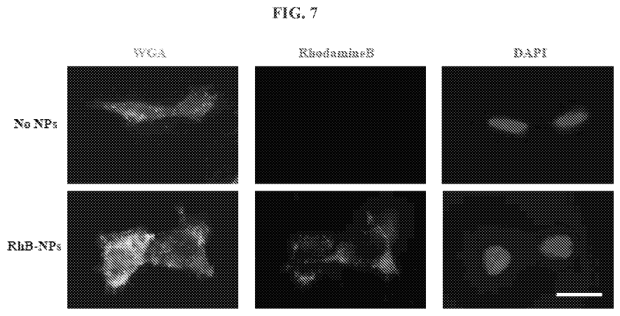

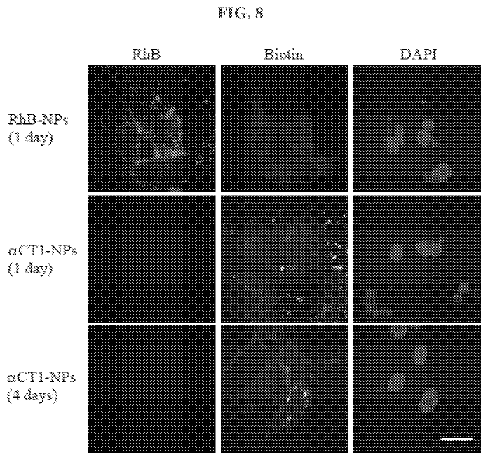

Staining of cell membranes and nuclei with and without RhB-NPs added at 200 μg/ml in the medium of LN229/GSCs shows cellular uptake of NPs is occurring, as RhB-NPs are present in the cytosol but excluded from the nuclei (FIG. 7). When αCT1-NPs were added at 1 mg/ml in the medium of SF295 cells (RhB-NPs at 1 mg/ml used as a positive control for NP cellular uptake), detection of biotin-tagged αCT1 following αCT1-NP uptake demonstrates the presence of αCT1 in the cells after one and four days (FIG. 8).

### DISCUSSION

This Example describes a method to encapsulate the peptide αCT1 using a double emulsion-solvent evaporation method a well as the methods used to minimize particle size for the purpose of sterilization of particles via filtration. Particles release peptide over about 21 days in vitro. It is also demonstrated that the particles are ingested by VTC-037 GSCs and the presence of peptide in cells is detectable for at least four days. The results indicate that particles may be used for controlled release of αCT1 peptide.

One factor to consider when creating implantable materials is the sterilization process. Here, sterilization by filtration was determined to be unlikely to alter the release profile of the encapsulated peptide, so particle size was optimized to achieve the smallest diameter. Particle size optimization reduced the average particle size from 229 nm in diameter to 143 nm in diameter. All other particle sizes had average diameters under 170 nm. This is likely due to dropping the primary emulsion into the PVA solution while vortexing, which became possible when a smaller batch size was used. Smaller batch sizes prevent the solutions from being thrown out of the container during vortexing when the primary emulsion is added to the secondary outer phase.

Sample 3 showed the favorable conditions for minimalizing particle diameter during size optimization study.

Single and double emulsions may encapsulate amounts of drug differently. For example, single emulsions generally encapsulate hydrophobic drugs in higher loading percentages, while double emulsions provide a space for hydrophilic drugs to occupy so they are less driven towards the outermost phase.²¹ The RhB content in the particles here are similar between the single and double emulsions. Because so little RhB was actually encapsulated, the low loading could be mostly due to adsorption on the surface of the particles rather than incorporation inside the particles. Encapsulation efficiency may be higher for the double emulsion particles because more particles were collected than for single emulsion particles.

Increases in NP size over time was anticipated due to fact that PLGA generally erodes in bulk at neutral to acidic pH at small thicknesses.²⁷ Bulk erosion occurs when degradation speed occurs more slowly than water uptake, compared with surface erosion in which degradation and removal of polymer occurs more quickly than water uptake. In the case of surface erosion, degradation is limited to the surface of the polymer matrix.^{27, 28} Here, bulk-eroding PLGA-NPs swell with water during the first few days while the polymer degrades hydrolytically but before erosion occurs.^{27, 29} When the particles increase in size again around three weeks, agglomeration of the particles is likely the main cause of size increase. Rescignano et al. also observed agglomeration in nanoparticles as degradation progressed.³⁰ This is likely due to PVA stabilizer being removed from the surface during nanoparticle erosion. PVA, which is likely incorporated into the PLGA shell even after wash cycles, helps sterically hinder agglomeration. PVA dissolves in water, so as the particle degrades the PVA could also be dislodging and dissolving into the surrounding water. Once the steric hindrance is removed, it would be easier for the particles to agglomerate, thus the size increases. The holes that appear in the NPs are likely due to polymer being removed through erosion channels from the particles during degradation and erosion.^{29, 30}

When NPs were introduced to cells, staining of cell membranes and nuclei with and without RhB-NPs (FIG. 7) RhB-NPs were detected in the cytosol but excluded from the nuclei, supporting NP cellular uptake. When cells are incubated at 4° C., little cellular uptake of the RhB-NPs occurred, which implies energy-dependent endocytosis is the main pathway of NP uptake.^{31, 32} RhB-NPs as well as αCT1-NPs are internalized by the cells.

In conclusion, PLGA nanoparticles successfully encapsulated αCT1 and released 73% of the drug over three weeks in vitro. When introduced to human GSCs, RhB was clearly detectable for the first seven days, and then at minimal amounts at 14 and 21 days. RhB was likely detected as a released RhB molecule as wells as still encapsulated in a particle. αCT1-NPs introduced to GSC showed αCT1 present in cells over at least four days. However, drug loading and encapsulation efficiency were low, below the therapeutically relevant doses of αCT1, unless an impractical amount of nanoparticles were to be used. Example 2 details further studies which improved drug loading in order to raise the dosage to more effective levels for patients.

### Example 2: Preparation of αCT1-Loaded PLGA Nanoparticles by Flash Nanoprecipitation

The double emulsion technique (water/oil/water emulsion) suffered drawbacks such as αCT1-NP loading profiles (˜1% encapsulation efficiency), whereas single emulsion synthesis NPs showed a tendency to aggregate; resulting in the need to expand this aim to explore additional synthesis techniques. Additional techniques which were investigated include: i.) the optimal nontoxic organic solvent that evaporates quickly while efficiently dissolves the polymer but not αCT1; ii.) application of a rotovap to expedite solvent removal to lessen time for the peptide to diffuse into the outer aqueous phase; iii.) the addition of zinc oxide or calcium into the outer phase to increase encapsulation efficiency and create a dense, homogeneous sphere. Lyophilization and freezing techniques aimed at preserving nanoparticles for extended storage and transport, critical features for clinical translation and product commercialization, were also optimized. The addition of sucrose as a cryoprotectant during freezing and lyophilizing protocols successfully prevented particle aggregation.

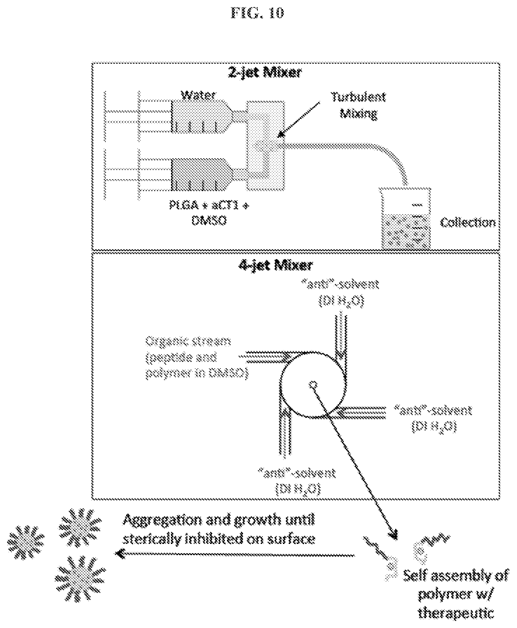

Flash nanoprecipitation provided the optimal αCT1-NPs in terms of higher drug loading and smaller, more consistent, unaggregated nanoparticles. This method involves dissolving the polymer (PLGA) and drug (αCT1) in a water-miscible solvent. Water was used as an “anti-solvent”, wherein the solvent used to dissolve the polymer is also miscible with the anti-solvent, but the polymer does not dissolve in the anti-solvent. When the two solvents are mixed, the polymer precipitates out of solution, capturing the drug in the process. Both a 2-jet mixer and 4-jet mixer approach were evaluated (FIG. 10).

Characterization of αCT1-NP size, morphology and surface charge, and determination of αCT1 loading and release efficiency. The following characterization parameters were completed for αCT1 and control NPs, and support the flash nanoprecipitation method (using the 4 jet mixer and >1% polyvinyl alcohol (PVA) (w/v) during mixing) for αCT1-NP for clinical development and commercialization: nanoparticle size and morphology were studied using scanning electron microscopy (SEM); efficiency of αCT1 encapsulation; and efficiency and release of αCT1 and rhodamine B.

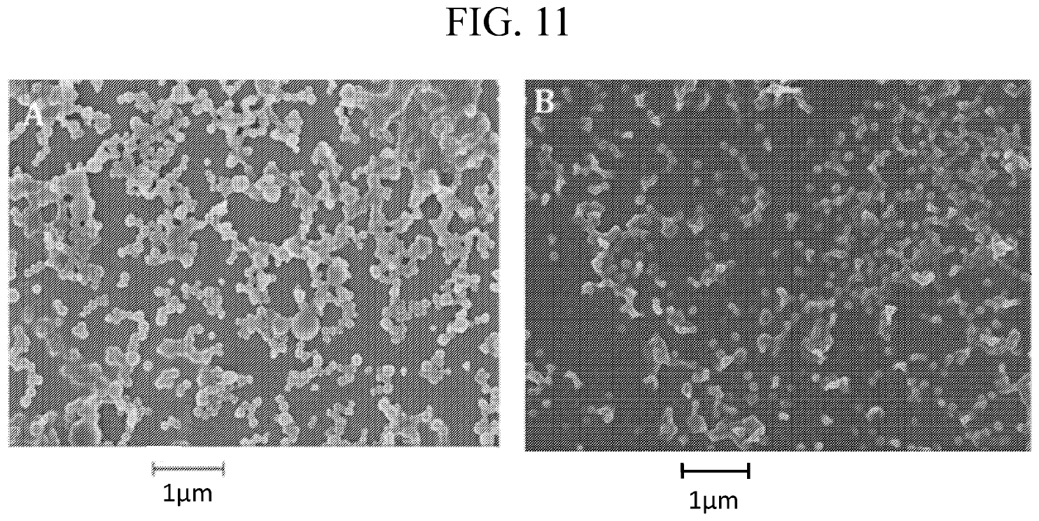

Nanoparticle size and morphology. Nanoparticles made via flash nanoprecipitation using the 4-jet mixer and PVA during mixing showed more consistent αCT1-NP size at −180 nm and less agglomeration vs the other methods. Nanoparticle size and morphology were studied using scanning electron microscopy (SEM). This is important because a homogenous distribution in morphology and size will result in nanoparticles with optimized physico-chemical properties and more consistent results regarding αCT1 release. Particle size was measured with a Zetasizer Nano ZS using dynamic light scattering techniques. The optimal nanoparticle size for loading efficiency and cellular uptake is 100-300 nm and <150 nm+\−40 for CED delivery. αCT1-NPs showed smooth surface morphology without noticeable pinholes or cracks, with average sizes from 100-200 nm (FIG. 11). The addition of PVA during mixing (0.3-2.5%) resulted in smaller particle size.

Overall, Flash nanoprecipitation using the 4-jet mixer and PVA during mixing showed more consistent αCT1-NP size at ˜180 nm and less agglomeration vs the other methods.

The encapsulation efficiency was determined by measuring the αCT1 peptide content and rhodamine B content of digested particles. For this purpose, a known quantity of lyophilized nanoparticles was resuspended in DMSO and the solution was agitated at 50° C. for 30 min before adding 50% acetonitrile with 0.1% trifluoroacetic acid for an extra 60 min to allow the degradation of PLGA. Degraded PLGA was eliminated by centrifugation and the supernatant containing αCT1 and rhodamine B was analyzed using a newly developed and optimized ELISA and fluorescence quantification, respectively. A highly sensitive and specific (>90%) sandwich ELISA using a C-terminal Cx43 antibody (1 μg/ml) for capture and HRP-tagged Neutravidin for detection, was specifically developed and validated for the purposes of these studies. The concentration of αCT1 in the nanoparticles was calculated using a calibration curve created with known concentrations of αCT1 or rhodamine B. αCT1 drug loading was optimal (˜65.7%) when flash nanoprecipation synthesis with the 4-jet mixer was used. Where the addition of >1% PVA during mixing significantly increased loading efficiency. The addition of sodium chloride did not noticeably impact drug loading efficiency.

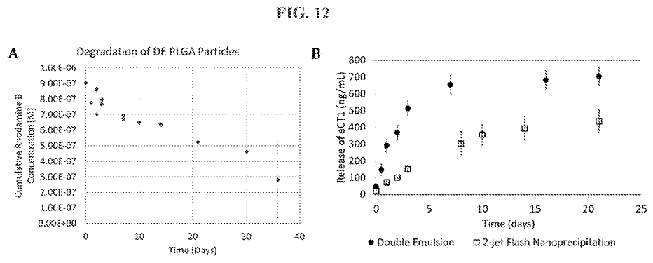

The in vitro release rate of encapsulated αCT1 and rhodamine B from the NPs was determined by incubating in PBS solution at 37° C. with constant mixing by magnetic stirrer. At regular time intervals from 0 h to 36 days, samples of the suspension were collected, and fresh PBS was added to maintain a constant volume in the nanoparticle suspension. Each collected sample was centrifuged to eliminate NPs, and the concentration of αCT1 and rhodamine B in the supernatant was determined as described above. Degradation studies showed maintained PLGA-Rhod-NP integrity for >36 days (FIG. 12A). Analyses of αCT1 by double emulsion release showed an initial burst after 1 day followed by a sustained release with a maximum reached after 7 days. Particles were redispersed in PBS at a concentration of 1 mg/mL and degraded at 37° C. The 2-jet flash nanoprecipitation method shows less of a burst release compared with the double emulsion particles; likely due to the inclusion of a 24-hr dialysis step used remove solvent (FIG. 12B).

Thus, flash nanoprecipitation afforded higher αCT1 encapsulation and better αCT1 release kinetics compared to other methods.

### Example 3: Cellular Uptake of αCT1-NP

Analysis of αCT1-NP cellular uptake in GBM cells. Cellular uptake of αCT1-NPs was evaluated in human GBM cells in vitro. The presence of αCT1-NP inside the cells was assessed using confocal microscopy by detection of i.) rhodamine B (presence of nanoparticles) and the ii.) αCT1 peptide. Different concentrations (ranging from 20-300 μg/ml) of rhodamine B labeled αCT1-NP were added to the media of human GBM and normal astrocyte cell lines. The cells were washed 5× with PBS and fixed at different time points from 1 to 24 and analyzed by fluorescence microscopy. Fluorescent wheat germ agglutinin (WGA) Alexa Fluor® 488 was used to stain cell membrane, identifying the perimeter of each cell; and DAPI was used to stain the nuclei.

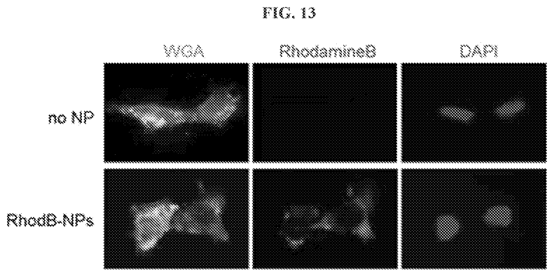

LN229/GSCs showed strong uptake of RhodB-NPs after 24 hours (FIG. 13). Similarly, in cultured GSCs (named VTC-037) that were isolated from a glioblastoma patient who received surgery at the Carilion Clinic, strong RhodB-NP uptake was observed at 1 hour and 24 hours (data not shown due space limitations). Cellular uptake was diminished at 4° C. Uptake of Rhod-NP by GSC neurospheres derived from GBM patients showed uptake at higher concentrations (>250 μg/ml), where isolation and dissociation of cells showed about 50-60% of cells positive for Rhod-NPs. These studies confirm that PLGA NPs showed optimal GBM cellular uptake via endocytosis at NP concentrations above 75 μg/ml and are also able to penetrate into GSC neurospheres. Interestingly, normal human astrocytes showed PLGA-NP uptake at concentrations >500 μg/ml.

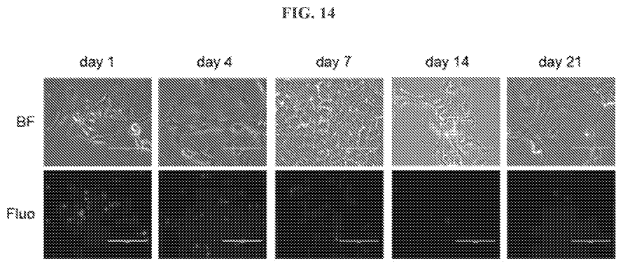

To evaluate in vitro NP degradation, Rhod-NPs (300 μg/ml) were added to VTC-037 cells for 24 hrs and observed by florescence microscopy for 21 days. Cells were passaged through trypsinisation at day 10. Rhodamine signal was still detected after 3 weeks of cell culture, and after passaging; thus supporting the integrity of the PLGA NPs for >21 days (FIG. 14).

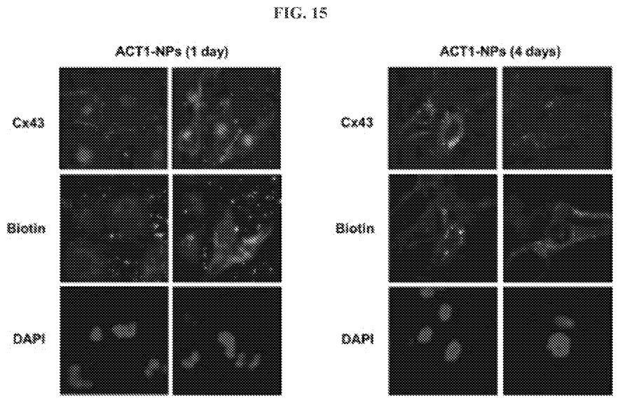

αCT1 comprises an antennapedia cell penetration domain that promotes cellular uptake. Therefore, the degradation of αCT1-NP outside the cells will result in availability and uptake of αCT1 into the cells. Staining using Alexa Fluor® 647 Streptavidin was used to evaluate the presence of biotin-tagged αCT1 in the cells using confocal microscopy and confirm the efficiency of the antennapedia domain to internalize αCT1 peptide. Following exposure to αCT1-NPs for one day prior to washing and adding fresh media, αCT1 could be detected in vitro in human GBM cells for at least 4 days (FIG. 15). Staining with a C terminal Cx43 antibody positively confirmed αCT1 staining.

### Example 4. Therapeutic Application of αCT1-NP

The in vivo therapeutic potential of αCT1-loaded biodegradable particles in sensitizing brain tumors to TMZ treatment was assessed. Evaluation of αCT1-NP biodistribution in the mouse brain following microinjection and analyses of the therapeutic potential of αCT1-NP in combination with TMZ in xenograft GBM model was conducted.

Results: Biodegradable, sterile, αCT1-loaded PLGA nanoparticles delivered via microinjection were detected close to the injection site and were not associated with toxicity. Intratumoral delivery of αCT-NP in conjunction with TMZ treatment showed efficacy in significantly reducing tumor volume in a GBM mouse model.

Evaluation of αCT1-NP distribution in mouse brain. Biotin-tagged αCT1-NP labeled with rhodamine B (1 mg/ml concentration) was infused (3 μl) in mouse brains using a microinjection pump. Non-labeled empty NPs were infused as negative control. Mouse brains were harvested at 1 day and 1 week. Brains were collected and sucrose cryoprotected before freezing after OCT embedding. In addition, blood and tissue from other organs including lungs, heart, and liver, were collected for additional analyses. Detection of Rhod-NPs in brain sections close to the injection site after 1 day and 1 week was seen (data not shown due to space limitations). Critically no adverse events were detected in treated mice. This included no changes in neurobiobehavioral evaluation, nor evidence of neuronal or systemic toxicity associated with αCT1 PLGA-NP delivery.

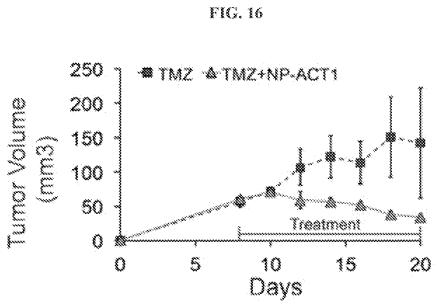

Effect of αCT1-NP in a xenograft GBM mouse model. To assess the in vivo safety and efficacy of αCT1-NPs, a mouse xenograft model of GBM was employed U87MG GBM cells were cultured and 1×106 cells were mixed with 100 μL of Matrigel® Matrix and subcutaneously injected into the flanks of SCID/beige anesthetized mice using 23 gauge needles. After 8 days, mice were divided into 2 groups: (i) TMZ alone, and (ii) TMZ+αCT1 particles. The treatment regimen was as follows: TMZ (7.5 mg/kg) was administered by intraperitoneal injection with an insulin syringe every other day starting on day 8 for both groups 1 and 2 mice. αCT1 particles were reconstituted in 1×PBS from −80° C. storage and 100 uL aliquots were prepared and stored at −20° C. The concentration of αCT1 delivered in 1 dose of particles (100 uL) was approximately 8 μM at 1.2 mg particles/kg of mouse body weight. αCT1 particles were delivered at the tumor growth site every other day starting on day 10 for group 2 mice using insulin syringes. Group 2 mice received a total of 6 injections of particles during the course of treatment. This treatment regimen continued for 13 days. Tumor sizes were measured every other day using a caliper. Tumor volume was calculated ((length×width2)/2). Treatment with TMZ+αCT1-NP resulted in a significant reduction in tumor volume (FIG. 16).

Results from PLGA-NP formulation development, and in vitro and in vivo delivery studies in ex vivo models support the feasibility developing αCT1-NPs for sustained and controlled delivery of αCT1 to GBM cells, and the therapeutic potential of αCT1-NPs.

In summary, a highly sensitive and specific αCT1 (>90%) sandwich ELISA using a C-terminal Cx43 antibody (1 μg/ml) for capture and HRP-tagged Neutravidin for detection was specifically developed and validated. Biodegradable αCT1-loaded poly(lactic-co-glycolic acid) (PLGA) nanoparticles (NPs) were developed, optimized and validated, specifically with characteristics necessary for targeted convection-enhanced delivery (CED) in GBM patients (FDA approved copolymer; <150 nm+\−40 in diameter, controlled and sustained (>3 weeks) αCT1 release profile). It was confirmed that αCT1 loaded nanoparticles (αCT1-NP) are efficiently taken up by human GMB cell lines as well as GSC neurospheres and NP can be detected in >21 days. In vivo biodistribution studies in rodents confirmed extended NP presence in the brain close to the injection site for at least one week. The efficacy of αCT1-NPs was validated in vivo in a mouse xenograft GBM model, where αCT-NP in combination with TMZ significantly reduced GBM tumor progression.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

All documents cited or otherwise referenced or disclosed herein are incorporated by reference in their entirety for all purposes.

### REFERENCES

1. M. H. Xiong, Y. Bao, X. Z. Yang, Y. H. Zhu and J. Wang, Delivery of antibiotics with polymeric particles. Adv Drug Deliv Rev. 2014; 78:63-76
2. T. Akagi, M. Baba and M. Akashi, Biodegradable Nanoparticles as Vaccine Adjuvants and Delivery Systems: Regulation of Immune Responses by Nanoparticle-Based Vaccine. 2011; 247:31-64
3. J. M. Anderson and M. S. Shive, Biodegradation and biocompatibility of PLA and PLGA microspheres. Advanced Drug Delivery Reviews. 1997; 28:19
4. F. Danhier, E. Ansorena, J. M. Silva, R. Coco, A. Le Breton and V. Preat, PLGA-based nanoparticles: an overview of biomedical applications. J Control Release. 2012; 161:505-22
5. K. E. Uhrich, S. M. Cannizzaro, R. S. Langer and K. M. Shakesheff, Polymeric Systems for Controlled Drug Release. Chemical Reviews. 1999; 99:3181-3198
6. G. S. Ghatnekar, C. L. Grek, D. G. Armstrong, S. C. Desai and R. G. Gourdie, The effect of a connexin43-based Peptide on the healing of chronic venous leg ulcers: a multicenter, randomized trial. J Invest Dermatol. 2015; 135:289-298
7. C. L. Grek, G. M. Prasad, V. Viswanathan, D. G. Armstrong, R. G. Gourdie and G. S. Ghatnekar, Topical administration of a connexin43-based peptide augments healing of chronic neuropathic diabetic foot ulcers: A multicenter, randomized trial. Wound Repair Regen. 2015; 23:203-12
8. ClinicalTrials.gov, 2016. A Study of Granexin Gel to Treat Diabetic Foot Ulcer, NIH: U.S. National Library of Medicine,
9. S. F. Murphy, R. T. Varghese, S. Lamouille, S. Guo, K. J. Pridham, P. Kanabur, et al., Connexin 43 Inhibition Sensitizes Chemoresistant Glioblastoma Cells to Temozolomide. Cancer Res. 2016; 76:139-49
10. K. Moore, J. Amos, J. Davis, R. Gourdie and J. D. Potts, Characterization of polymeric microcapsules containing a low molecular weight peptide for controlled release. Microsc Microanal. 2013; 19:213-26
11. K. Moore, Z. J. Bryant, G. Ghatnekar, U. P. Singh, R. G. Gourdie and J. D. Potts, A synthetic connexin 43 mimetic peptide augments corneal wound healing. Exp Eye Res. 2013; 115:178-88
12. A. Mathew, T. Fukuda, Y. Nagaoka, T. Hasumura, H. Morimoto, Y. Yoshida, et al., Curcumin loaded-PLGA nanoparticles conjugated with Tet-1 peptide for potential use in Alzheimer's disease. PLoS One. 2012; 7:e32616
13. K. K. Chereddy, C. H. Her, M. Comune, C. Moia, A. Lopes, P. E. Porporato, et al., PLGA nanoparticles loaded with host defense peptide LL37 promote wound healing. J Control Release. 2014; 194:138-47
14. M. S. Cartiera, E. C. Ferreira, C. Caputo, M. E. Egan, M. J. Caplan and W. M. Saltzman, Partial correction of cystic fibrosis defects with PLGA nanoparticles encapsulating curcumin. Mol Pharm. 2010; 7:86-93
15. M. Morales-Cruz, G. M. Flores-Femandez, M. Morales-Cruz, E. A. Orellano, J. A. Rodriguez-Martinez, M. Ruiz, et al., Two-step nanoprecipitation for the production of protein-loaded PLGA nanospheres. Results Pharma Sci. 2012; 2:79-85
16. Q. Liu, X. Chen, J. Jia, W. Zhang, T. Yang, L. Wang, et al., pH-Responsive Poly(D,L-lactic-co-glycolic acid) Nanoparticles with Rapid Antigen Release Behavior Promote Immune Response. ACS Nano. 2015; 9:14
17. X. Zhang, G. Chen, L. Wen, F. Yang, A. L. Shao, X. Li, et al., Novel multiple agents loaded PLGA nanoparticles for brain delivery via inner ear administration: in vitro and in vivo evaluation. Eur J Pharm Sci. 2013; 48:595-603
18. X. Zhang, M. Sun, A. Zheng, D. Cao, Y. Bi and J. Sun, Preparation and characterization of insulin-loaded bioadhesive PLGA nanoparticles for oral administration. Eur J Pharm Sci. 2012; 45:632-8
19. H. K. Makadia and S. J. Siegel, Poly Lactic-co-Glycolic Acid (PLGA) as Biodegradable Controlled Drug Delivery Carrier. Polymers (Basel). 2011; 3:1377-1397
20. S. S. L. Peppin, M. G. Worster and J. S. Wettlaufer, Morphological instability in freezing colloidal suspensions. Proceedings of the Royal Society A: Mathematical, Physical and Engineering Sciences. 2007; 463:723-733
21. P. Fonte, S. Soares, A. Costa, J. C. Andrade, V. Seabra, S. Reis, et al., Effect of cryoprotectants on the porosity and stability of insulin-loaded PLGA nanoparticles after freeze-drying. Biomatter. 2012; 2:329-39
22. K. S. Tang, S. M. Hashmi and E. M. Shapiro, The effect of cryoprotection on the use of PLGA encapsulated iron oxide nanoparticles for magnetic cell labeling. Nanotechnology. 2013; 24:125101
23. S. Bozdag, K. Dillen, J. Vandervoort and A. Ludwig, The effect of freeze-drying with different cryoprotectants and gamma-irradiation sterilization on the characteristics of ciprofloxacin HCl-loaded poly(D,L-lactide-glycolide) nanoparticles. J Pharm Pharmacol. 2005; 57:699-707
24. T. Musumeci, C. A. Ventura, I. Giannone, B. Ruozi, L. Montenegro, R. Pignatello, et al., PLA/PLGA nanoparticles for sustained release of docetaxel. Int J Pharm. 2006; 325:172-9 R. A. Jain, The manufacturing techniques of various drug loaded biodegradable poly(lactide-co-glycolide) (PLGA) devices. Biomaterials. 2000:16
25. A. Budhian, S. J. Siegel and K. I. Winey, Haloperidol-loaded PLGA nanoparticles: systematic study of particle size and drug content. Int J Pharm. 2007; 336:367-75
26. F. v. Burkersroda, L. Schedl and A. Gopferich, Why degradable polymers undergo surface erosion or bulk erosion. Biomaterials. 2002; 23:11
27. J. A. Tamada and R. Langer, Erosion kinetics of hydrolytically degradable polymers. Proc Natl Acad Sci USA. 1993; 90:5
28. A. Gopferich, Polymer Bulk Erosion. Macromolecules. 1997; 30:7
29. N. Rescignano, M. Amelia, A. Credi, J. M. Kenny and I. Armentano, Morphological and thermal behavior of porous biopolymeric nanoparticles. European Polymer Journal. 2012; 48:1152-1159
30. R. Firdessa, T. A. Oelschlaeger and H. Moll, Identification of multiple cellular uptake pathways of polystyrene nanoparticles and factors affecting the uptake: relevance for drug delivery systems. Eur J Cell Biol. 2014; 93:323-37
31. L. Kou, J. Sun, Y. Zhai and Z. He, The endocytosis and intracellular fate of nanomedicines: Implication for rational design. Asian Journal of Pharmaceutical Sciences. 2013; 8:1-10

1. A composition comprising one or more nanoparticles, wherein the nanoparticles comprise one or more biodegradable or biocompatible polymers and a therapeutically effective amount of a peptide comprising an amino acid sequence according to SEQ ID NO: 1.

2. The composition of claim 1, wherein the peptide further comprises a cellular internalization sequence.

3. The composition of claim 2, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

4. The composition of any one of claims 1 to 3, wherein the peptide comprises an amino acid sequence according to SEQ ID NO: 2.

5. The nanoparticle composition of any one of claims 1-4, wherein the one or more biodegradable or biocompatible polymers are selected from the group consisting of poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyethylene, polypropylene, poly(ethylene glycol) (PEG), polyalkylene oxides (PEO), poly(ethylene terephthalate), polyvinyl alcohols (PVA), polyvinyl ethers, poly(vinyl acetate), poly(vinyl chloride) (PVC), polyvinylpyrrolidone (PVP), polysiloxanes, polystyrene, polyurethanes, alkyl celluloses, hydroxyalkyl celluloses, cellulose ethers, cellulose esters, nitro celluloses, poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate), polydioxanone, polydioxanone copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

6. The nanoparticle composition of claim 5, wherein the one or more biodegradable or biocompatible polymers are PLGA.

7. The nanoparticle composition of claim 6, wherein the one or more biodegradable or biocompatible polymers are PLGA and PVA.

8. The nanoparticle composition of any one of the preceding claims, wherein the average diameter of the nanoparticles is between about 10 nm and about 1000 nm.

9. The nanoparticle composition of claim 8, wherein the average diameter of the nanoparticles is between about 30 nm and about 500 nm.

10. The nanoparticle composition of claim 9, wherein the average diameter of the nanoparticles is between about 100 nm and about 300 nm.

11. The nanoparticle composition of claim 10, wherein the average diameter of the nanoparticles is between about 100 nm and about 200 nm.

12. The nanoparticle composition of claim 11, wherein the average diameter of the nanoparticles is about 180 nm.

13. The nanoparticle composition of claim 6 or 7, wherein the PLGA has a Mw from about 7,000 to about 17,000 Da.

14. The nanoparticle composition of claim 6, 7, or 13, wherein the PLGA is 50:50 lactic acid:glycolic acid, acid terminated.

15. The nanoparticle composition of claim 7 or claim 8, wherein the PVA has a Mw from about 13,000 to about 23,000 Da.

16. The nanoparticle composition of claim 7, wherein the amount of PVA is between about 0.05% (w/v) to about 5% (w/v).

17. The nanoparticle composition of claim 8, wherein the amount of PVA is between about 0.3% (w/v) to about 2.5% (w/v).

18. The nanoparticle composition of claim 6, wherein the amount of PLGA is between about 2% (w/v) to about 10% (w/v).

19. The nanoparticle composition of claim 6, wherein the average amount of the peptide comprising an amino acid sequence according to SEQ ID NO: 1 or SEQ ID NO: 2 is at least about 500 ng per mg of the nanoparticle composition.

20. The nanoparticle composition of any one of the preceding claims, further comprising a cryoprotectant.

21. The nanoparticle composition of claim 20, wherein the cryoprotectant is sucrose, trehalose, dextrose, or sorbitol.

22. The nanoparticle composition of any one of the preceding claims, wherein the nanoparticles have a surface charge characterized by a zeta potential of between about 0 mV to about −30 mV.

23. The nanoparticle composition of any one of the preceding claims, wherein the nanoparticles have a polydispersity index (PDI) of from about 0.120 to about 0.350.

24. The nanoparticle composition of any one of the preceding claims, wherein the nanoparticles further comprise zinc oxide or calcium.

25. The nanoparticle composition of any one of the preceding claims, wherein the nanoparticles have a peptide load greater than about 10 ng peptide/μg.

26. The nanoparticle composition of claim 25, wherein the nanoparticles have a peptide load greater than about 100 ng peptide/μg particles.

27. The nanoparticle composition of any of the preceding claims, wherein the nanoparticles exhibit a controlled peptide release profile substantially corresponding to the following pattern:
after 24 hours, from about 5% and 60% of the total encapsulated peptide is released; and
after 21 days, from about 50% to 100% of the total encapsulated peptide is released.

28. A pharmaceutical formulation comprising the nanoparticle composition of any one of claims 1-27 and one or more pharmaceutically acceptable carriers or excipients.

29. The pharmaceutical formulation of claim 28, wherein the formulation is a liquid formulated for injection.

30. The pharmaceutical formulation of claim 28 or 29, wherein the one or more pharmaceutically acceptable carriers or excipients are sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

31. The pharmaceutical formulation of any one of claims 28-30, further comprising diluents such as buffers, antioxidants such as ascorbic acid, carbohydrates such as glucose, sucrose or dextrins, chelating agents such as EDTA, and glutathione.

32. The pharmaceutical formulation of claim 28, wherein the one or more pharmaceutically acceptable carriers or excipients are selected from the groups consisting of anti-adherents, antioxidants, binders, coatings, compression aids, disintegrants, dyes (colors), emollients, emulsifiers, fillers (diluents), film formers or coatings, flavors, glidants (flow enhancers), lubricants, preservatives, sorbents, suspending or dispersing agents, sweeteners, and waters of hydration.

33. The pharmaceutical formulation of claim 28 or 32, wherein the one or more pharmaceutically acceptable carriers or excipients are selected from the groups consisting of butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, and xylitol.

34. The pharmaceutical formulation of claim 28, wherein the formulation is in the form of an aerosol, cream, foam, emulsion, gel, liquid, lotion, patch, powder, solid, spray, or any combinations thereof.

35. The pharmaceutical formulation of claim 28, formulated for administration by subcutaneous, intradermal, intramuscular, intratumoral, or intravenous injection.

36. A topical formulation comprising the nanoparticle composition of any one of claims 1-27.

37. The topical formulation of claim 36, further comprising a buffering agent.

38. The topical formulation of claim 37, wherein the buffering agent is a phosphate buffer.

39. A method of making the nanoparticle composition of claims 1-27, comprising the steps of
(a) combining a first solution comprising one or more biodegradable or biocompatible polymers dissolved in an organic solvent with a second solution comprising an amino acid sequence according to SEQ ID NO: 1 or SEQ ID NO: 2 dissolved in a first aqueous solvent;
(b) emulsifying the mixture of step (a);
(c) adding the emulsion of step (b) to a second solution comprising one or more biodegradable or biocompatible polymers dissolved in a second aqueous solvent;
(d) removing the organic solvent; and
(e) optionally purifying the product of (d).

40. The method of claim 39, further comprising the step of (f) freezing and/or lyophilizing the product of (d) or (e).

41. The method of claim 39 or claim 40, wherein step (d) is performed by stirring the mixture of (c) for an amount of time sufficient to remove the organic solvent from the mixture.

42. The method of claim 41, wherein the amount of time sufficient to remove the organic solvent from the mixture is from about 1 minute to about 10 hours.

43. The method of claim 42, wherein the amount of time sufficient to remove the organic solvent from the mixture is about 3 hours.

44. The method of claim 39 or claim 40, wherein step (d) is performed by rotary evaporation.

45. The method of any one of claims 39-44, wherein step (a) is performed while being sonicated.

46. The method of any one of claims 39-44, wherein step (c) is performed while being vortexted.

47. The method of any one of claims 39-46, wherein the product of step (c) is sonicated prior to step (d).

48. The method of any one of claims 39-47, wherein the purifying of step (e) is performed by washing the product of (d).

49. The method of any one of claims 39-48, wherein the product of any one of steps (c)-(f) are sterilized.

50. The method of any one of claims 39-49, wherein the biodegradable or biocompatible polymers of step (a) is PLGA.

51. The method of any one of claims 39-50, wherein the biodegradable or biocompatible polymers of step (c) further comprises PVA.

52. The method of any one of claims 39-51, wherein the organic solvent is ethyl acetate.

53. The method of any one of claims 39-52, wherein the first aqueous solvent and second aqueous solvent are water.

54. A method of making the nanoparticle composition of claims 1-27, comprising
(a) providing
i. an amino acid sequence according to SEQ ID NO: 1 or SEQ ID NO: 2 and one or more biodegradable or biocompatible polymers dissolved in a water-miscible organic solvent; and
ii. an anti-solvent;
(b) mixing amino acid sequence according to SEQ ID NO: 1 or SEQ ID NO: 2 and one or more biodegradable or biocompatible polymers dissolved in the water-miscible organic solvent with the anti-solvent, such that the nanoparticle composition is formed; and
(c) optionally purifying the product of (b).

55. The method of claim 54, further comprising the step of
(d) freezing and/or lyophilizing the product of (b) or (c).

56. The method of claim 54, wherein the mixing is performed with a jet mixer.

57. The method of claim 56, wherein the jet mixer is a 2-jet mixer or a 4-jet mixer.

58. The method of claim 57, wherein the jet mixer is a 2-jet mixer.

59. The method of claim 57, wherein the jet mixer is a 4-jet mixer.

60. The method of any one of claims 39-59, wherein the water-miscible organic solvent is DMSO.

61. The method of any one of claims 39-60, wherein the anti-solvent is water.

62. The method of any one of claims 39-61, wherein the biodegradable or biocompatible polymers of step (a) is PLGA.

63. The method of claim 62, wherein the biodegradable or biocompatible polymers of step (a) further comprises PVA.

64. The method of any one of claims 54-63, wherein the nanoparticle encapsulation efficiency of the amino acid sequence is greater than about 20%.

65. The method of any one of claims 54-63, wherein the nanoparticle encapsulation efficiency of the amino acid sequence is greater than about 65%.

66. A method of manufacturing a topical formulation comprising:
a) mixing propylene glycol, glycerin, methylparaben and propylparaben until the parabens are completely dissolved;
b) separately mixing purified water, EDTA, monobasic sodium phosphate, dibasic sodium phosphate and D-mannitol until a clear solution is obtained;
c) adding the solution from a) to the solution from b), rinsing the container of the solution from a) with purified water, adding the rinse to the combined solutions, and mixing until the combined solutions are visually homogeneous;
d) with homogenization mixing, adding hydroxyethyl cellulose into the combined solutions of c) and mixing until the polymer is fully dispersed;
e) separately mixing purified water with an amino acid sequence according to SEQ ID NO: 1 or SEQ ID NO: 2 until the peptide is completely dissolved;
f) adding the solution from e) to the solution of d), rinsing the container of the solution from e) with purified water, adding the rinse to the combined solutions, and mixing until the combined solution are homogeneous.

67. A method of treating cancer in a patient in need thereof wherein the method comprises, administering to the patient a therapeutically effective amount of the pharmaceutical formulation of claims 28-35.

68. The method of claim 67, wherein the cancer is selected from the group consisting of glioma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma, glioblastoma, ovarian cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers, testicular cancer, colon and rectal cancers, prostatic cancer, pancreatic cancer, rhabdomyosarcoma, spinal cord tumors, and bone cancers such as osteosarcoma, Ewing's sarcoma, chondrosarcoma, multiple myeloma and giant-cell bone tumor. In some embodiments, the cancer is glioma. In some embodiments, the glioma is glioblastoma.

69. The method of claim 68, wherein the cancer is glioma.

70. The method of claim 69, wherein the glioma is glioblastoma.

71. The method of any one of claims 67-70, wherein the pharmaceutical formulation is administered to the patient by subcutaneous, intradermal, intramuscular, intratumoral, or intravenous injection.

72. The method of claim 71, wherein the pharmaceutical formulation is administered by intratumoral injection.

73. The method of any one of claims 67-72, further comprising administering a chemotherapeutic agent.

74. The method of claim 73, wherein the chemotherapeutic agent is selected from the group consisting of saporin, ricin, abrin, ethidium bromide, diptheria toxin, Pseudomonas exotoxin, temozolomide (TMZ), chlorambucil, cyclophosphamide, isofamide, mechlorethamine, melphalan, uracil mustard, thiotepa, busulfan, carmustine, lomustine, streptozocin, cisplatin, carboplatin, tetraplatin, ormaplatin, thioplatin, satraplatin, nedaplatin, oxaliplatin, heptaplatin, iproplatin, transplatin, lobaplatin, mitomycin, procarbazine, dacarbazine, altretamine, bleomycin, amsacrine, menogaril, amonafide, dactinomycin, daunorubicin, N,N-dibenzyl daunomycin, ellipticine, daunomycin, pyrazoloacridine, idarubicin, mitoxantrone, m-AMSA, bisantrene, doxorubicin (adriamycin), deoxydoxorubicin, etoposide (VP-16), etoposide phosphate, oxanthrazole, rubidazone, epirubicin, bleomycin, teniposide, plicamydin, methotrexate, trimetrexate, fluorouracil, fluorodeoxyuridine, CB3717, azacitidine, cytarabine, floxuridine, mercaptopurine, 6-thioguanine, fludarabine, pentostatin, asparginase, hydroxyurea, vincristine, vinblastine, and paclitaxel (Taxol®).

75. The method of claim 73 or claim 74, wherein the chemotherapeutic agent is not administered concomitantly with the pharmaceutical formulations.

76. The method of claim 75, wherein the chemotherapeutic agent is administered on a different day than the pharmaceutical formulation.

77. A method of treating a chronic wound in a subject, comprising administering to the subject the topical formulation of claims 36-38, wherein the formulation is administered in a dosing regimen effective for the treatment of the chronic wound.
